(19) United States
(12) Patent Application Publication
(10) Pub. No.: US 2010/0331715 A1
(43) Pub. Date: Dec. 30, 2010

(54) SYSTEMS AND METHODS FOR DETECTING EFFORT EVENTS

(75) Inventors: Paul Stanley Addison, Edinburgh (GB); Andrew Cassidy, Edinburgh (GB); James N. Watson, Dunfermline (GB); Scott McGonigle, Edinburgh (GB)

Correspondence Address: Nellcor Puritan Bennett LLC;ATTN: IP Legal, 6135 Gunbarrel Avenue, Boulder, CO, 80301, US

(73) Assignee: Nellcor Puritan Bennett Ireland, Mervue (IE)

(21) Appl. No.: 12/495,018

(22) Filed: Jun. 30, 2009

(51) Int. Cl. A61B 5/08; A61B 5/113

(52) U.S. Cl. 600/529

(57) ABSTRACT

A method and system for detecting effort events is disclosed. Effort may be determined through feature analysis of the signal as transformed by a continuous wavelet transform, which may be compared against a reference effort measure to trigger an effort event flag that signals the onset and/or severity of an effort event. For example, a respiratory effort measure may be determined based at least in part on a wavelet transform of a photoplethysmograph (PP G) signal and features of the transformed signal. A respiratory reference effort measure may be based at least in part on past values of the respiratory effort measure, and a threshold test may be used to trigger an effort event flag, which may indicate a marked change in respiratory effort exerted by a patient.

### SUMMARY OF THE DISCLOSURE

The present disclosure relates to patient monitoring and, more particularly, the present disclosure relates to using physiological effort signals, such as those derived from a continuous wavelet transform of a photoplethysmograph (PPG) signal, to detect physiological effort events.

A physiological effort event may be any significant status or change in status of the physiological exertion of a patient. When a patient is undergoing physiological monitoring, effort events may be manifest in characteristics of the monitored signals that indicate a decrease or increase in effort. For example, the cessation of normal breathing activity (e.g., an apneic event) may be identified by detecting an irregularity in one or more of a number of physiological signals, including the rise and fall of a patients chest during respiration as measured by a transducer attached to a chest or abdominal strap; temperature changes in a patients nasal or oral cavities as measured by a thermocouple, or pressure/airflow changes measured by, for example, one or more transducers in the respiratory tract.

However, each of these approaches may be limited in its ability to correctly detect and/or classify an apneic or other respiratory effort event. For example, a patient's chest may continue to rise and fall during an obstructive apneic event, though little or no air may be flowing and respiratory effort has increased. Additionally, a thermocouple used to detect airflow may exhibit decreased sensitivity at higher levels of airflow, reducing its ability to detect a hypopnea event or an increase in respiratory effort (e.g., a hyperpneic event). Accordingly, there is a need for methods and systems for monitoring physiological effort signals that detect effort events and may flag a user or care provider to such events.

One measure of effort is based on a scalogram derived from a continuous wavelet transform of a monitored signal. Techniques for deriving such effort measures are described in detail in Addison et al., U.S. application Ser. No. 12/245,366, filed Oct. 3, 2008, entitled “Systems and Methods for Determining Effort,” which is incorporated by reference herein in its entirety. These effort measures may be based on techniques for identifying the energy content of features within a scalogram associated with respiration.

In some embodiments, the use of a transform may allow a signal to be represented in a suitable domain such as, for example, a scalogram (in a time-scale domain) or a spectrogram (in a time-frequency domain). A type of effort which may be determined by analyzing the signal representation may be, for example, the respiratory effort of a patient. The determination of effort from a scalogram or any other signal representation is possible because changes in effort induce or change various features of the signal used to generate the scalogram. For example, the act of breathing may cause a breathing band to become present in a scalogram that was derived from a PPG signal. This band may occur at or about the scale having a characteristic frequency that corresponds to the breathing frequency. Furthermore, the features within this band or other bands in the scalogram (e.g., energy, amplitude, phase, or modulation) may result from changes in breathing and/or breathing effort and therefore may be correlated with the patient's breathing effort.

In this disclosure, methods and systems are provided for using physiological effort information to detect and flag significant physiological events. These physiological events may include respiratory events that represent an increase or decrease in respiratory effort, such as apnea, hypopnea, or changes in effort due to the administration of a drug (e.g., a bronchodilator). Effort may be determined through feature analysis of the signal as transformed by a continuous wavelet transform, and may be compared against a reference effort measure to trigger an effort event flag that signals the onset and/or severity of an effort event. For example, a respiratory effort measure may be determined based at least in part on a wavelet transform of a photoplethysmograph (PPG) signal and features of the transformed signal. A respiratory reference effort measure may be based at least in part on past values of the respiratory effort measure, and a threshold test may, for example, be used to trigger an effort event flag, which may indicate a marked change in respiratory effort exerted by a patient.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The above and other features of the present disclosure, its nature and various advantages will be more apparent upon consideration of the following detailed description, taken in conjunction with the accompanying drawings in which:

FIG. 1 shows an illustrative effort system in accordance with an embodiment;

FIG. 2 is a block diagram of the illustrative effort system of FIG. 1 coupled to a patient in accordance with an embodiment;

FIGS. 3(a) and 3(b) show illustrative views of a scalogram derived from a PPG signal in accordance with an embodiment;

FIG. 3(c) shows an illustrative scalogram derived from a signal containing two pertinent components in accordance with an embodiment;

FIG. 3(d) shows an illustrative schematic of signals associated with a ridge of the scalogram of FIG. 3(c) and illustrative schematics of a further wavelet decomposition of these signals in accordance with an embodiment;

FIGS. 3(e) and 3(f) are flow charts of illustrative steps involved in performing an inverse continuous wavelet transform in accordance with an embodiment;

FIG. 4 is a block diagram of an illustrative continuous wavelet processing system in accordance with an embodiment;

FIG. 5 is an illustrative scalogram showing the manifestation of a plurality of bands and an increase in effort in accordance with an embodiment;

FIG. 6 is a flow chart depicting illustrative steps used to determine effort in accordance with some embodiments;

FIG. 7 depicts illustrative data representative of respiratory effort in accordance with an embodiment;

FIG. 8 is a flow chart depicting illustrative steps used to generate an effort event flag in accordance with an embodiment;

FIG. 9(a) depicts an illustrative effort signal and an effort event determination process in accordance with an embodiment;

FIG. 9(b) depicts an illustrative effort monitoring system display screen in accordance with an embodiment; and

FIG. 10 depicts an illustrative band of a scalogram and several effort measures that may be used to determine an effort event in accordance with an embodiment.

### DETAILED DESCRIPTION

An oximeter is a medical device that may determine the oxygen saturation of the blood. One common type of oximeter is a pulse oximeter, which may indirectly measure the oxygen saturation of a patient's blood (as opposed to measuring oxygen saturation directly by analyzing a blood sample taken from the patient) and changes in blood volume in the skin. Ancillary to the blood oxygen saturation measurement, pulse oximeters may also be used to measure the pulse rate of the patient. Pulse oximeters typically measure and display various blood flow characteristics including, but not limited to, the oxygen saturation of hemoglobin in arterial blood. Pulse oximeters may also be used to determine respiratory effort in accordance with the present disclosure.

An oximeter may include a light sensor that is placed at a site on a patient, typically a fingertip, toe, forehead or earlobe, or in the case of a neonate, across a foot. The oximeter may pass light using a light source through blood perfused tissue and photoelectrically sense the absorption of light in the tissue. For example, the oximeter may measure the intensity of light that is received at the light sensor as a function of time. A signal representing light intensity versus time or a mathematical manipulation of this signal (e.g., a scaled version thereof, a log taken thereof, a scaled version of a log taken thereof, etc.) may be referred to as the photoplethysmograph (PPG) signal. In addition, the term “PPG signal,” as used herein, may also refer to an absorption signal (i.e., representing the amount of light absorbed by the tissue) or any suitable mathematical manipulation thereof. The light intensity or the amount of light absorbed may then be used to calculate the amount of the blood constituent (e.g., oxyhemoglobin) being measured as well as the pulse rate and when each individual pulse occurs.

The light passed through the tissue is selected to be of one or more wavelengths that are absorbed by the blood in an amount representative of the amount of the blood constituent present in the blood. The amount of light passed through the tissue varies in accordance with the changing amount of blood constituent in the tissue and the related light absorption. Red and infrared (JR) wavelengths may be used because it has been observed that highly oxygenated blood will absorb relatively less Red light and more IR light than blood with a lower oxygen saturation. By comparing the intensities of two wavelengths at different points in the pulse cycle, it is possible to estimate the blood oxygen saturation of hemoglobin in arterial blood.

When the measured blood parameter is the oxygen saturation of hemoglobin, a convenient starting point assumes a saturation calculation based at least in part on Lambert-Beer's law. The following notation will be used herein:

I(λ, t)=I₀(λ)exp(−(sβ₀(λ)+(1−s)βᵣ(λ))l(t)) (1)

where:
λ=wavelength;
t=time;
I=intensity of light detected;
I₀ intensity of light transmitted;
s=oxygen saturation;
β₀, βᵣ=empirically-derived absorption coefficients; and
l(t)=a combination of concentration and path length from emitter to detector as a function of time.

The traditional approach measures light absorption at two wavelengths (e.g., Red and IR), and then calculates saturation by solving for the “ratio of ratios” as follows.
1. The natural logarithm of Eq. 1 is taken (“log” will be used to represent the natural logarithm) for IR and Red to yield

log I=log I₀−(sβ₀+(1−s)βᵣ)l (2)
2. Eq. 2 is then differentiated with respect to time to yield

$$\frac{d log I}{d t} = - ( s {β}_{o} + ( 1 - s ) {β}_{r} ) \frac{d l}{d t} . \qquad ( 3 )$$

3. Eq. 3, evaluated at the Red wavelength AR is divided by Eq. 3 evaluated at the IR wavelength IR in accordance with

$$\frac{d log I ( {λ}_{R} ) \text{/} d t}{d log I ( {λ}_{IR} ) \text{/} d t} = \frac{s {β}_{o} ( {λ}_{R} ) + ( 1 - s ) {β}_{r} ( {λ}_{R} )}{s {β}_{o} ( {λ}_{IR} ) + ( 1 - s ) {β}_{r} ( {λ}_{IR} )} . \qquad ( 4 )$$

4. Solving for s yields

$$s = \frac{\frac{d log I ( {λ}_{IR} )}{d t} {β}_{r} ( {λ}_{R} ) - \frac{d log I ( {λ}_{R} )}{d t} {β}_{r} ( {λ}_{IR} )}{\begin{matrix}\frac{d log I ( {λ}_{R} )}{d t} ( {β}_{o} ( {λ}_{IR} ) - {β}_{r} ( {λ}_{IR} ) ) - \\ \frac{d log I ( {λ}_{IR} )}{d t} ( {β}_{o} ( {λ}_{R} ) - {β}_{r} ( {λ}_{R} ) )\end{matrix}} . \qquad ( 5 )$$

5. Note that, in discrete time, the following approximation can be made:

$$\frac{d log I ( λ , t )}{d t} ≃ log I ( λ , {t}_{2} ) - log I ( λ , {t}_{1} ) \qquad ( 6 )$$

6. Rewriting Eq. 6 by observing that log A−log B=log(A/B) yields

$$\frac{d log I ( λ , t )}{d t} ≃ log ( \frac{I ( {t}_{2} , λ )}{I ( {t}_{1} , λ )} ) . \qquad ( 7 )$$

7. Thus, Eq. 4 can be expressed as

$$\frac{\frac{d log I ( {λ}_{R} )}{d t}}{\frac{d log I ( {λ}_{IR} )}{d t}} ≃ \frac{log ( \frac{I ( {t}_{1} , {λ}_{R} )}{I ( {t}_{2} , {λ}_{R} )} )}{log ( \frac{I ( {t}_{1} , {λ}_{IR} )}{I ( {t}_{2} , {λ}_{IR} )} )} = R . \qquad ( 8 )$$

where R represents the “ratio of ratios.”
8. Solving Eq. 4 for s using the relationship of Eq. 5 yields

$$s = \frac{{β}_{r} ( {λ}_{R} ) - R {β}_{r} ( {λ}_{IR} )}{R ( {β}_{o} ( {λ}_{IR} ) - {β}_{r} ( {λ}_{IR} ) ) - {β}_{o} ( {λ}_{R} ) + {β}_{r} ( {λ}_{R} )} . \qquad ( 9 )$$

9. From Eq. 8, R can be calculated using two points (e.g, PPG maximum and minimum), or a family of points. One method applies a family of points to a modified version of Eq. 8. Using the relationship

$$\frac{d log I}{d t} = \frac{d I \text{/} d t}{I} , \qquad ( 10 )$$

Eq. (8) becomes

$$\begin{matrix}\frac{\frac{d log I ( {λ}_{R} )}{d t}}{\frac{d log I ( {λ}_{IR} )}{d t}} ≃ \frac{\frac{I ( {t}_{2} , {λ}_{R} ) - I ( {t}_{1} , {λ}_{R} )}{I ( {t}_{1} , {λ}_{R} )}}{\frac{I ( {t}_{2} , {λ}_{IR} ) - I ( {t}_{1} , {λ}_{IR} )}{I ( {t}_{1} , {λ}_{IR} )}} \\ = \frac{[ I ( {t}_{2} , {λ}_{R} ) - I ( {t}_{1} , {λ}_{R} ) ] I ( {t}_{1} , {λ}_{IR} )}{[ I ( {t}_{2} , {λ}_{IR} ) - I ( {t}_{1} , {λ}_{IR} ) ] I ( {t}_{1} , {λ}_{R} )} \\ = R ,\end{matrix} \qquad ( 11 )$$

which defines a cluster of points whose slope of y versus x will give R when

x=[I(t₂, λ_{IR})−I(t₁, λ_{IR})]I(t₁, λ_{R}) (12)

and

y=[I(t₂, λ_{R})−I(t₁, λ_{R})]I(t₁, λ_{IR}). (13)

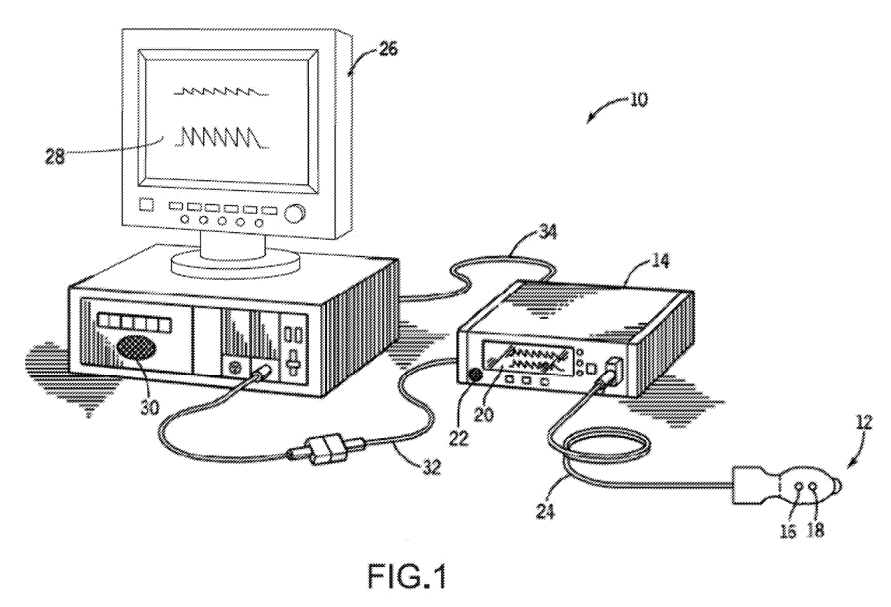

FIG. 1 is a perspective view of an embodiment of an effort system 10. In an embodiment, effort system 10 is implemented as part of a pulse oximetry system. System 10 may include a sensor 12 and a monitor 14. Sensor 12 may include an emitter 16 for emitting light at two or more wavelengths into a patient's tissue. A detector 18 may also be provided in sensor 12 for detecting the light originally from emitter 16 that emanates from the patient's tissue after passing through the tissue.

Sensor 12 or monitor 14 may further include, but are not limited to software modules that calculate respiration rate, airflow sensors (e.g., nasal thermistor), ventilators, chest straps, transthoracic sensors (e.g., sensors that measure transthoracie impedance).

According to another embodiment and as will be described, system 10 may include a plurality of sensors forming a sensor array in lieu of single sensor 12. Each of the sensors of the sensor array may be a complementary metal oxide semiconductor (CMOS) sensor. Alternatively, each sensor of the array may be a charged coupled device (CCD) sensor. In another embodiment, the sensor array may be made up of a combination of CMOS and CCD sensors. The CCD sensor may comprise a photoactive region and a transmission region for receiving and transmitting data whereas the CMOS sensor may be made up of an integrated circuit having an array of pixel sensors. Each pixel may have a photodetector and an active amplifier.

According to an embodiment, emitter 16 and detector 18 may be on opposite sides of a digit such as a finger or toe, in which case the light that is emanating from the tissue has passed completely through the digit. In an embodiment, emitter 16 and detector 18 may be arranged so that light from emitter 16 penetrates the tissue and is reflected by the tissue into detector 18, such as a sensor designed to obtain pulse oximetry data from a patient's forehead.

In an embodiment, the sensor or sensor array may be connected to and draw its power from monitor 14 as shown. In another embodiment, the sensor may be wirelessly connected to monitor 14 and include its own battery or similar power supply (not shown). Monitor 14 may be configured to calculate physiological parameters based at least in part on data received from sensor 12 relating to light emission and detection. In an alternative embodiment, the calculations may be performed on the monitoring device itself and the result of the effort or oximetry reading may be passed to monitor 14. Further, monitor 14 may include a display 20 configured to display a patient's physiological parameters or other information about the system. In the embodiment shown, monitor 14 may also include a speaker 22 to provide an audible sound that may be used in various other embodiments, such as, for example, sounding an audible alarm in the event that a patient's physiological parameters are not within a predefined normal range.

In an embodiment, sensor 12, or the sensor array, may be communicatively coupled to monitor 14 via a cable 24. However, in other embodiments, a wireless transmission device (not shown) or the like may be used instead of or in addition to cable 24.

In the illustrated embodiment, effort system 10 may also include a multi-parameter patient monitor 26. The monitor may be cathode ray tube type, a flat panel display (as shown) such as a liquid crystal display (LCD) or a plasma display, or any other type of monitor now known or later developed. Multi-parameter patient monitor 26 may be configured to calculate physiological parameters and to provide a display 28 for information from monitor 14 and from other medical monitoring devices or systems (not shown). For example, multi-parameter patient monitor 26 may be configured to display an estimate of a patient's respiratory effort or blood oxygen saturation (referred to as an “SpO₂” measurement) generated by monitor 14, pulse rate information from monitor 14 and blood pressure from a blood pressure monitor (not shown) on display 28.

Monitor 14 may be communicatively coupled to multi-parameter patient monitor 26 via a cable 32 or 34 that is coupled to a sensor input port or a digital communications port, respectively, and/or may communicate wirelessly (not shown). In addition, monitor 14 and/or multi-parameter patient monitor 26 may be coupled to a network to enable the sharing of information with servers or other workstations (not shown). Monitor 14 may be powered by a battery (not shown) or by a conventional power source such as a wall outlet.

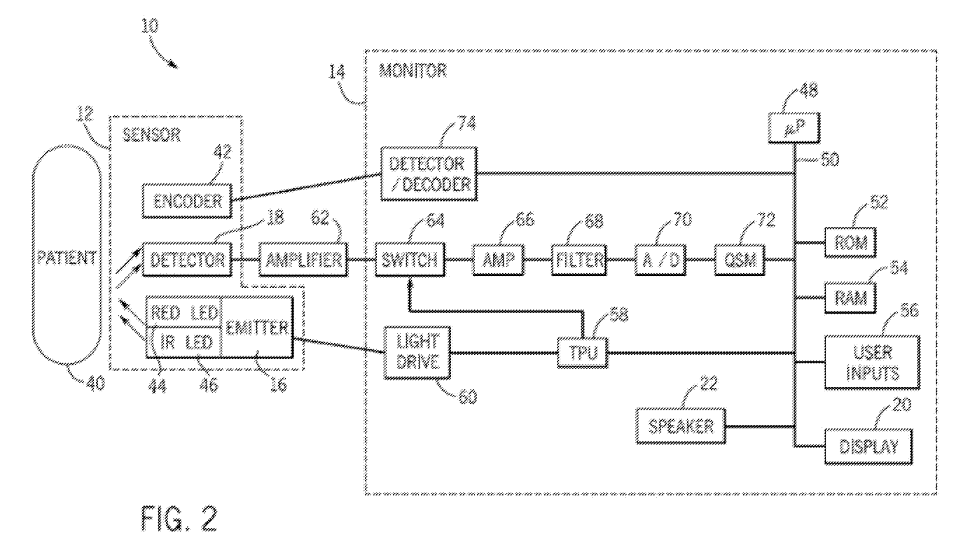

FIG. 2 is a block diagram of an effort system, such as effort system 10 of FIG. 1, which may be coupled to a patient 40 in accordance with an embodiment. Certain illustrative components of sensor 12 and monitor 14 are illustrated in FIG. 2. Sensor 12 may include emitter 16, detector 18, and encoder 42. In the embodiment shown, emitter 16 may be configured to emit one or more wavelengths of light (e.g., Red and/or IR) into a patient's tissue 40. Hence, emitter 16 may include a Red light emitting light source such as Red light emitting diode (LED) 44 and/or an IR light emitting light source such as IR LED 46 for emitting light into the patient's tissue 40 at the wavelengths used to calculate the patient's physiological parameters. In one embodiment, the Red wavelength may be between about 600 nm and about 700 nm, and the IR wavelength may be between about 800 nm and about 1000 nm. In embodiments in which a sensor array is used in place of a single sensor, each sensor may be configured to emit a single wavelength. For example, a first sensor emits only a Red light while a second only emits an IR light.

It will be understood that, as used herein, the term “light” may refer to energy produced by radiative sources and may include one or more of ultrasound, radio, microwave, millimeter wave, infrared, visible, ultraviolet, gamma ray or X-ray electromagnetic radiation. As used herein, light may also include any wavelength within the radio, microwave, infrared, visible, ultraviolet, or X-ray spectra, and that any suitable wavelength of electromagnetic radiation may be appropriate for use with the present techniques. Detector 18 may be chosen to be specifically sensitive to the chosen targeted energy spectrum of the emitter 16.

In an embodiment, detector 18 may be configured to detect the intensity of light at the Red and IR wavelengths. Alternatively, each sensor in the array may be configured to detect an intensity of a single wavelength. In operation, light may enter detector 18 after passing through the patient's tissue 40. Detector 18 may convert the intensity of the received light into an electrical signal. The light intensity is directly related to the absorbance and/or reflectance of light in the tissue 40. That is, when more light at a certain wavelength is absorbed or reflected, less light of that wavelength is received from the tissue by the detector 18. After converting the received light to an electrical signal, detector 18 may send the signal to monitor 14, where physiological parameters may be calculated based on the absorption of the Red and IR wavelengths in the patient's tissue 40.

In an embodiment, encoder 42 may contain information about sensor 12, such as what type of sensor it is (e.g., whether the sensor is intended for placement on a forehead or digit) and the wavelength or wavelengths of light emitted by emitter 16. This information may be used by monitor 14 to select appropriate algorithms, lookup tables and/or calibration coefficients stored in monitor 14 for calculating the patient's physiological parameters.

Encoder 42 may contain information specific to patient 40, such as, for example, the patient's age, weight, and diagnosis. This information may allow monitor 14 to determine, for example, patient-specific threshold ranges in which the patient's physiological parameter measurements should fall and to enable or disable additional physiological parameter algorithms. Encoder 42 may, for instance, be a coded resistor which stores values corresponding to the type of sensor 12 or the type of each sensor in the sensor array, the wavelengths of light emitted by emitter 16 on each sensor of the sensor array, and/or the patient's characteristics. In an embodiment, encoder 42 may include a memory on which one or more of the following information may be stored for communication to monitor 14: the type of the sensor 12, the wavelengths of light emitted by emitter 16, the particular wavelength each sensor in the sensor array is monitoring, a signal threshold for each sensor in the sensor array, any other suitable information, or any combination thereof.

In an embodiment, signals from detector 18 and encoder 42 may be transmitted to monitor 14. In the embodiment shown, monitor 14 may include a general-purpose microprocessor 48 connected to an internal bus 50. Microprocessor 48 may be adapted to execute software, which may include an operating system and one or more applications, as part of performing the functions described herein. Also connected to bus 50 may be a read-only memory (ROM) 52, a random access memory (RAM) 54, user inputs 56, display 20, and speaker 22.

RAM 54 and ROM 52 are illustrated by way of example, and not limitation. Any suitable computer-readable media may be used in the system for data storage. Computer-readable media are capable of storing information that can be interpreted by microprocessor 48. This information may be data or may take the form of computer-executable instructions, such as software applications, that cause the microprocessor to perform certain functions and/or computer-implemented methods. Depending on the embodiment, such computer-readable media may include computer storage media and communication media. Computer storage media may include volatile and non-volatile, removable and non-removable media implemented in any method or technology for storage of information such as computer-readable instructions, data structures, program modules or other data. Computer storage media may include, but are not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by components of the system.

In the embodiment shown, a time processing unit (TPU) 58 may provide timing control signals to a light drive circuitry 60, which may control when emitter 16 is illuminated and multiplexed timing for the Red LED 44 and the IR LED 46. TPU 58 may also control the gating-in of signals from detector 18 through an amplifier 62 and a switching circuit 64. These signals are sampled at the proper time, depending upon which light source is illuminated. The received signal from detector 18 may be passed through an amplifier 66, a low pass filter 68, and an analog-to-digital converter 70. The digital data may then be stored in a queued serial module (QSM) 72 (or buffer) for later downloading to RAM 54 as QSM 72 fills up. In one embodiment, there may be multiple separate parallel paths having amplifier 66, filter 68, and A/ID converter 70 for multiple light wavelengths or spectra received.

In an embodiment, microprocessor 48 may determine the patient's physiological parameters, such as effort, SpO₂, and pulse rate, using various algorithms and/or look-up tables based on the value of the received signals and/or data corresponding to the light received by detector 18. Signals corresponding to information about patient 40, and particularly about the intensity of light emanating from a patient's tissue over time, may be transmitted from encoder 42 to a decoder 74. These signals may include, for example, encoded information relating to patient characteristics. Decoder 74 may translate these signals to enable the microprocessor to determine thresholds based on algorithms or look-up tables stored in ROM 52. User inputs 56 may be used to enter information about the patient, such as age, weight, height, diagnosis, medications, treatments, and so forth. Such information may be stored in a suitable memory (e.g., RAM 54) and may allow monitor 14 to determine, for example, patient-specific threshold ranges in which the patient's physiological parameter measurements should fall and to enable or disable additional physiological parameter algorithms. In an embodiment, display 20 may exhibit a list of values which may generally apply to the patient, such as, for example, age ranges or medication families, which the user may select using user inputs 56.

The optical signal through the tissue can be degraded by noise, among other sources. One source of noise is ambient light that reaches the light detector. Another source of noise is electromagnetic coupling from other electronic instruments. Movement of the patient also introduces noise and affects the signal. For example, the contact between the detector and the skin, or the emitter and the skin, can be temporarily disrupted when movement causes either to move away from the skin. In addition, because blood is a fluid, it responds differently than the surrounding tissue to inertial effects, thus resulting in momentary changes in volume at the point to which a probe is attached

Noise (e.g., from patient movement) can degrade a pulse oximetry signal relied upon by a physician, without the physician's awareness. This is especially true if the monitoring of the patient is remote, the motion is too small to be observed, or the doctor is watching the instrument or other parts of the patient and not the sensor site. Processing effort and pulse oximetry (i.e., PPG) signals may involve operations that reduce the amount of noise present in the signals or otherwise identify noise components in order to prevent them from affecting measurements of physiological parameters derived from the signals.

It will be understood that the present disclosure is applicable to any suitable signals and that PPG signals are used merely for illustrative purposes. Those skilled in the art will recognize that the present disclosure has wide applicability to other signals including, but not limited to other biosignals (e.g., electrocardiogram, electroencephalogram, electrogastrogram, electromyogram, heart rate signals, pathological sounds, ultrasound, or any other suitable biosignal), dynamic signals, non-destructive testing signals, condition monitoring signals, fluid signals, geophysical signals, astronomical signals, electrical signals, financial signals including financial indices, sound and speech signals, chemical signals, meteorological signals including climate signals, and/or any other suitable signal, and/or any combination thereof.

In one embodiment, a PPG signal may be transformed using a continuous wavelet transform. Information derived from the transform of the PPG signal (i.e., in wavelet space) may be used to provide measurements of one or more physiological parameters.

The continuous wavelet transform of a signal x(t) in accordance with the present disclosure may be defined as

$$T ( a , b ) = \frac{1}{\sqrt{a}} {\int }_{- ∞}^{+ ∞} x ( t ) {ψ}^{*} ( \frac{t - b}{a} ) d t \qquad ( 14 )$$

where ψ*(t) is the complex conjugate of the wavelet function ψ(t), a is the dilation parameter of the wavelet and b is the location parameter of the wavelet. The transform given by Eq. 14 may be used to construct a representation of a signal on a transform surface. The transform may be regarded as a time-scale representation. Wavelets are composed of a range of frequencies, one of which may be denoted as the characteristic frequency of the wavelet, where the characteristic frequency associated with the wavelet is inversely proportional to the scale a. One example of a characteristic frequency is the dominant frequency. Each scale of a particular wavelet may have a different characteristic frequency. The underlying mathematical detail required for the implementation within a time-scale can be found, for example, in Paul S. Addison, The Illustrated Wavelet Transform Handbook (Taylor & Francis Group 2002), which is hereby incorporated by reference herein in its entirety.

The continuous wavelet transform decomposes a signal using wavelets, which are generally highly localized in time. The continuous wavelet transform may provide a higher resolution relative to discrete transforms, thus providing the ability to garner more information from signals than typical frequency transforms such as Fourier transforms (or any other spectral techniques) or discrete wavelet transforms. Continuous wavelet transforms allow for the use of a range of wavelets with scales spanning the scales of interest of a signal such that small scale signal components correlate well with the smaller scale wavelets and thus manifest at high energies at smaller scales in the transform. Likewise, large scale signal components correlate well with the larger scale wavelets and thus manifest at high energies at larger scales in the transform. Thus, components at different scales may be separated and extracted in the wavelet transform domain. Moreover, the use of a continuous range of wavelets in scale and time position allows for a higher resolution transform than is possible relative to discrete techniques.

In addition, transforms and operations that convert a signal or any other type of data into a spectral (i.e., frequency) domain necessarily create a series of frequency transform values in a two-dimensional coordinate system where the two dimensions may be frequency and, for example, amplitude. For example, any type of Fourier transform would generate such a two-dimensional spectrum. In contrast, wavelet transforms, such as continuous wavelet transforms, are required to be defined in a three-dimensional coordinate system and generate a surface with dimensions of time, scale and, for example, amplitude. Hence, operations performed in a spectral domain cannot be performed in the wavelet domain; instead the wavelet surface must be transformed into a spectrum (i.e., by performing an inverse wavelet transform to convert the wavelet surface into the time domain and then performing a spectral transform from the time domain). Conversely, operations performed in the wavelet domain cannot be performed in the spectral domain; instead a spectrum must first be transformed into a wavelet surface (i.e., by performing an inverse spectral transform to convert the spectral domain into the time domain and then performing a wavelet transform from the time domain). Nor does a cross-section of the three-dimensional wavelet surface along, for example, a particular point in time equate to a frequency spectrum upon which spectral-based techniques may be used. At least because wavelet space includes a time dimension, spectral techniques and wavelet techniques are not interchangeable. It will be understood that converting a system that relies on spectral domain processing to one that relies on wavelet space processing would require significant and fundamental modifications to the system in order to accommodate the wavelet space processing (e.g, to derive a representative energy value for a signal or part of a signal requires integrating twice, across time and scale, in the wavelet domain while, conversely, one integration across frequency is required to derive a representative energy value from a spectral domain). As a further example, to reconstruct a temporal signal requires integrating twice, across time and scale, in the wavelet domain while, conversely, one integration across frequency is required to derive a temporal signal from a spectral domain. It is well known in the art that, in addition to or as an alternative to amplitude, parameters such as energy density, modulus, phase, among others may all be generated using such transforms and that these parameters have distinctly different contexts and meanings when defined in a two-dimensional frequency coordinate system rather than a three-dimensional wavelet coordinate system. For example, the phase of a Fourier system is calculated with respect to a single origin for all frequencies while the phase for a wavelet system is unfolded into two dimensions with respect to a wavelet's location (often in time) and scale.

The energy density function of the wavelet transform, the scalogram, is defined as

S(a, b)=|T(a, b)|² (15)

where ‘||’ is the modulus operator. The scalogram may be resealed for useful purposes. One common resealing is defined as

$${S}_{R} ( a , b ) = \frac{{\vert T ( a , b ) \vert}^{2}}{a} \qquad ( 16 )$$

and is useful for defining ridges in wavelet space when, for example, the Morlet wavelet is used. Ridges are defined as a locus of points of local maxima in the plane. Any reasonable definition of a ridge may be employed in the method. Also included as a definition of a ridge herein are paths displaced from the locus of the local maxima. A ridge associated with only the locus of points of local maxima in the plane is labeled a “maxima ridge.”

For implementations requiring fast numerical computation, the wavelet transform may be expressed as an approximation using Fourier transforms. Pursuant to the convolution theorem, because the wavelet transform is the cross-correlation of the signal with the wavelet function, the wavelet transform may be approximated in terms of an inverse FFT of the product of the Fourier transform of the signal and the Fourier transform of the wavelet for each required a scale and then multiplying the result by \sqrt{a}.

In the discussion of the technology which follows herein, a “scalogram” may be taken to include all suitable forms of rescaling including, but not limited to, the original unsealed wavelet representation, linear resealing, any power of the modulus of the wavelet transform, or any other suitable rescaling. In addition, for purposes of clarity and conciseness, the term “scalogram” shall be taken to mean the wavelet transform T(a, b) itself, or any part thereof. For example, the real part of the wavelet transform, the imaginary part of the wavelet transform, the phase of the wavelet transform, any other suitable part of the wavelet transform, or any combination thereof is intended to be conveyed by the term “scalogram.”

A scale, which may be interpreted as a representative temporal period, may be converted to a characteristic frequency of the wavelet function. The characteristic frequency associated with a wavelet of arbitrary a scale is given by

$$f = \frac{{f}_{c}}{a} \qquad ( 17 )$$

where f_{c} is the characteristic frequency of the mother wavelet (i.e., at a=1) and becomes a scaling constant, and f is the representative or characteristic frequency for the wavelet at arbitrary scale a.

Any suitable wavelet function may be used in connection with the present disclosure. One of the most commonly used complex wavelets, the Morlet wavelet, is defined as:

ψ(t)=π^{-1/4}(e^{i2xf}^{₀}^{t}−e^{-(2πf}^{₀}⁾^{²}_{/2})e^{-t}^{²}_{/2} (18)

where f₀ is the central frequency of the mother wavelet. The second term in the parentheses is known as the correction term, as it corrects for the non-zero mean of the complex sinusoid within the Gaussian window. In practice, it becomes negligible for values of f₀>>0 and can be ignored, in which case, the Morlet wavelet can be written in a simpler form as

$$ψ ( t ) = \frac{1}{{π}^{1 / 4}} {e}^{i 2 π {f}_{0} t} {e}^{- {t}^{2} / 2} . \qquad ( 19 )$$

This wavelet is a complex wave within a scaled Gaussian envelope. While both definitions of the Morlet wavelet are included herein, the function of Eq. 19 is not strictly a wavelet as it has a non-zero mean (i.e., the zero frequency term of its corresponding energy spectrum is non-zero) However, it will be recognized by those skilled in the art that Eq. 19 may be used in practice with f₀>>0 with minimal error and is included (as well as other similar near wavelet functions) in the definition of a wavelet herein. A more detailed overview of the underlying wavelet theory, including the definition of a wavelet function, can be found in the general literature. Discussed herein is how wavelet transform features may be extracted from the wavelet decomposition of signals. For example, wavelet decomposition of PPG signals may be used to provide clinically useful information within a medical device.

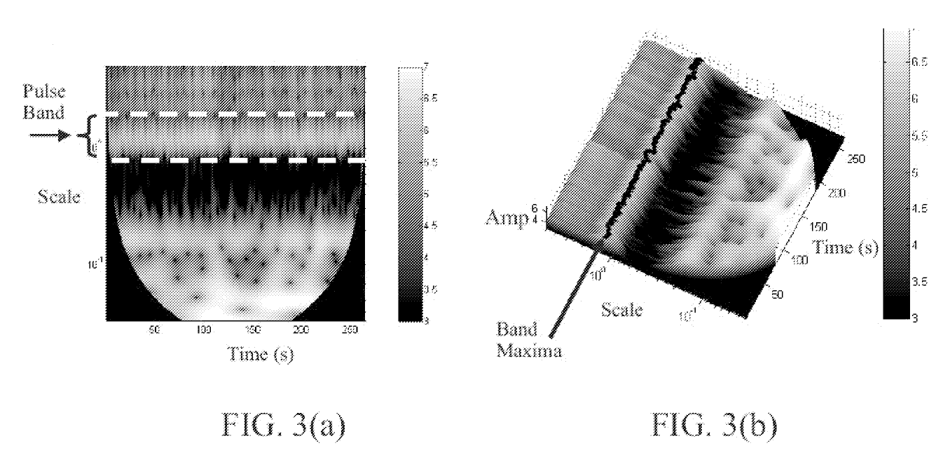

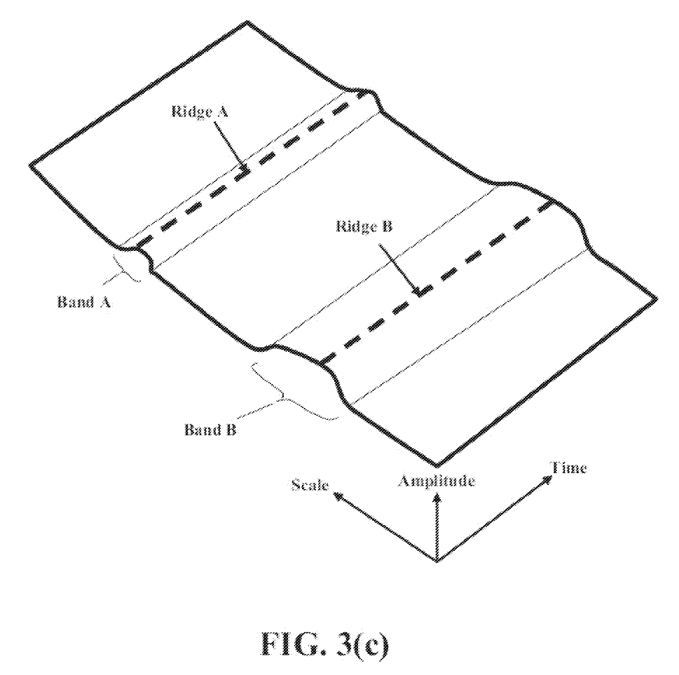

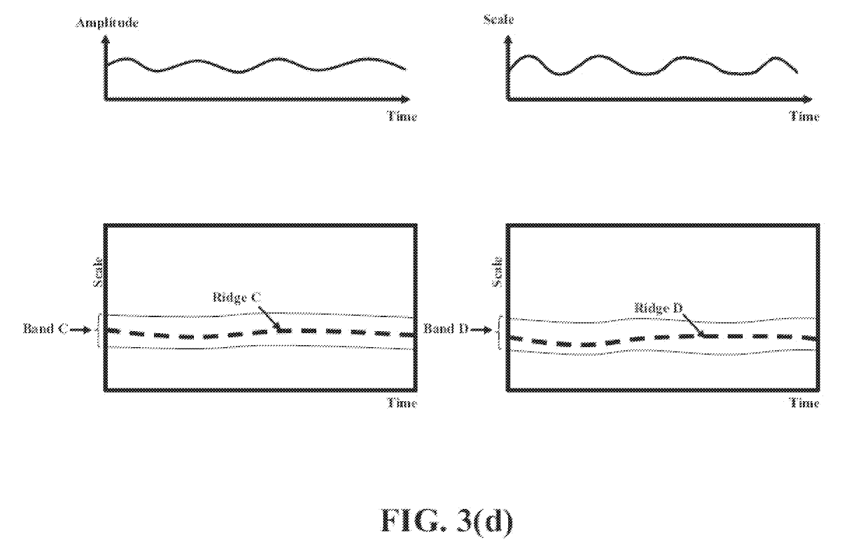

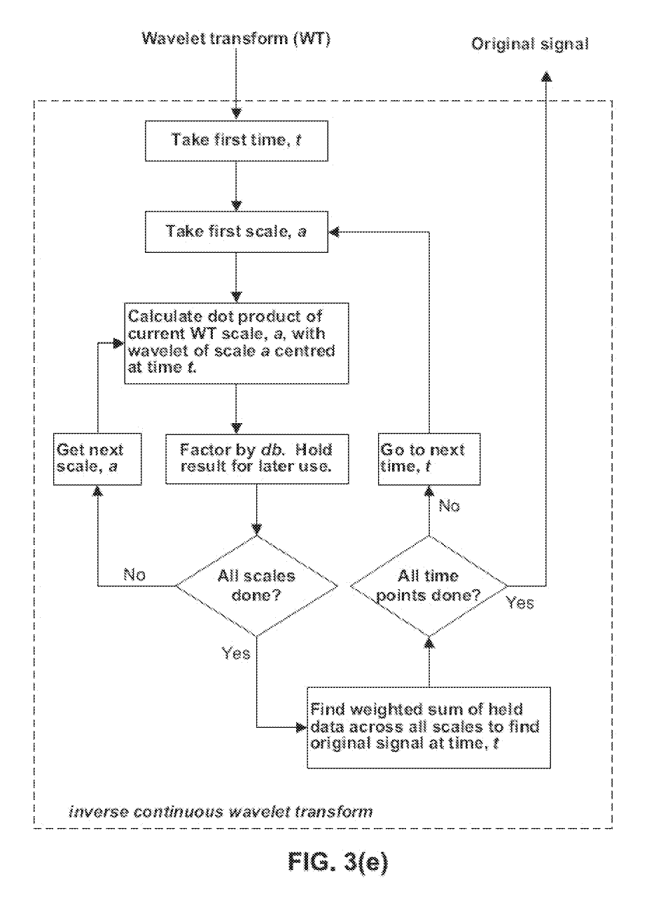

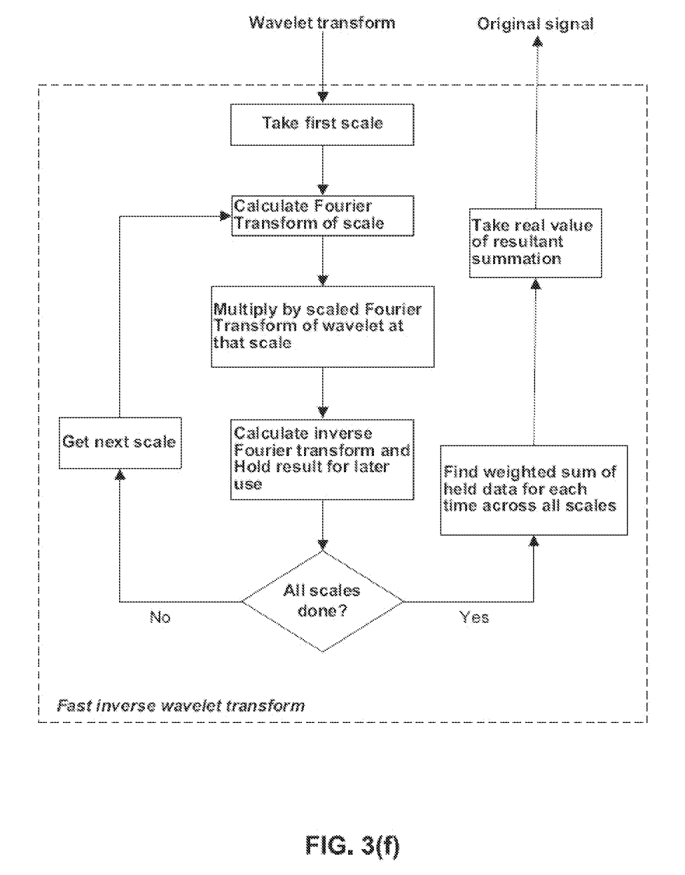

Pertinent repeating features in a signal give rise to a time-scale band in wavelet space or a resealed wavelet space. For example, the pulse component of a PPG signal produces a dominant band in wavelet space at or around the pulse frequency. FIGS. 3(a) and (b) show two views of an illustrative scalogram derived from a PPG signal, according to an embodiment. The figures show an example of the band caused by the pulse component in such a signal. The pulse band is located between the dashed lines in the plot of FIG. 3(a). The band is formed from a series of dominant coalescing features across the scalogram. This can be clearly seen as a raised band across the transform surface in FIG. 3(b) located within the region of scales indicated by the arrow in the plot (corresponding to 60 beats per minute). The maxima of this band with respect to scale is the ridge. The locus of the ridge is shown as a black curve on top of the band in FIG. 3(b). By employing a suitable resealing of the scalogram, such as that given in Eq. 16, the ridges found in wavelet space may be related to the instantaneous frequency of the signal. In this way, the pulse rate may be obtained from the PPG signal. Instead of resealing the scalogram, a suitable predefined relationship between the scale obtained from the ridge on the wavelet surface and the actual pulse rate may also be used to determine the pulse rate.

By mapping the time-scale coordinates of the pulse ridge onto the wavelet phase information gained through the wavelet transform, individual pulses may be captured. In this way, both times between individual pulses and the timing of components within each pulse may be monitored and used to detect heart beat anomalies, measure arterial system compliance, or perform any other suitable calculations or diagnostics. Alternative definitions of a ridge may be employed. Alternative relationships between the ridge and the pulse frequency of occurrence may be employed.

As discussed above, pertinent repeating features in the signal give rise to a time-scale band in wavelet space or a resealed wavelet space. For a periodic signal, this band remains at a constant scale in the time-scale plane. For many real signals, especially biological signals, the band may be non-stationary: varying in scale, amplitude, or both over time. FIG. 3(c) shows an illustrative schematic of a wavelet transform of a signal containing two pertinent components leading to two bands in the transform space, according to an embodiment. These bands are labeled band A and band B on the three-dimensional schematic of the wavelet surface. In an embodiment, a band ridge is defined as the locus of the peak values of these bands with respect to scale. For purposes of discussion, it may be assumed that band B contains the signal information of interest. Band B will be referred to as the “primary band.” In addition, it may be assumed that the system from which the signal originates, and from which the transform is subsequently derived, exhibits some form of coupling between the signal components in band A and band B. When noise or other erroneous features are present in the signal with similar spectral characteristics of the features of band B then the information within band B can become ambiguous (i.e., obscured, fragmented or missing). In this case the ridge of band A (referred to herein as “ridge A”) may be followed in wavelet space and extracted either as an amplitude signal or a scale signal which will be referred to as the “ridge amplitude perturbation” (RAP) signal and the “ridge scale perturbation” (RSP) signal, respectively. The RAP and RSP signals may be extracted by projecting the ridge onto the time-amplitude or time-scale planes, respectively. The top plots of FIG. 3(d) show a schematic of the RAP and RSP signals associated with ridge A in FIG. 3(c). Below these RAP and RSP signals are schematics of a further wavelet decomposition of these newly derived signals. This secondary wavelet decomposition allows for information in the region of band B in FIG. 3(c) to be made available as band C and band D. The ridges of bands C and D may serve as instantaneous time-scale characteristic measures of the signal components causing bands C and D. This technique, which will be referred to herein as secondary wavelet feature decoupling (SWFD), may allow information concerning the nature of the signal components associated with the underlying physical process causing the primary band B (FIG. 3(c)) to be extracted when band B itself is obscured in the presence of noise or other erroneous signal features.

In some instances, an inverse continuous wavelet transform may be desired, such as when modifications to a scalogram (or modifications to the coefficients of a transformed signal) have been made in order to, for example, remove artifacts. In one embodiment, there is an inverse continuous wavelet transform which allows the original signal to be recovered from its wavelet transform by integrating over all scales and locations, a and b, in accordance with

$$x ( t ) = \frac{1}{{C}_{g}} {\int }_{- ∞}^{∞} {\int }_{0}^{∞} T ( a , b ) \frac{1}{\sqrt{a}} ψ ( \frac{t - b}{a} ) \frac{d a d b}{{a}^{2}} , \qquad ( 20 )$$

which may also be written as

$$x ( t ) = \frac{1}{{C}_{g}} {\int }_{- ∞}^{∞} {\int }_{0}^{∞} T ( a , b ) {ψ}_{a , b} ( t ) \frac{d a d b}{{a}^{2}} . \qquad ( 21 )$$

where C_{g} is a scalar value known as the admissibility constant. It is wavelet-type dependent and may be calculated in accordance with

$${C}_{g} = {\int }_{0}^{∞} \frac{{\vert \hat{ψ} ( f ) \vert}^{2}}{f} d f . \qquad ( 22 )$$

FIG. 3(e) is a flow chart of illustrative steps that may be taken to perform an inverse continuous wavelet transform in accordance with the above discussion. An approximation to the inverse transform may be made by considering Eq. 20 to be a series of convolutions across scales. It shall be understood that there is no complex conjugate here, unlike for the cross correlations of the forward transform. As well as integrating over all of a and b for each time t, this equation may also take advantage of the convolution theorem which allows the inverse wavelet transform to be executed using a series of multiplications. FIG. 3(f) is a flow chart of illustrative steps that may be taken to perform an approximation of an inverse continuous wavelet transform. It will be understood that any other suitable technique for performing an inverse continuous wavelet transform may be used in accordance with the present disclosure.

The present disclosure relates to methods and systems for processing a signal using the above mentioned techniques, analyzing the results of the techniques to determine effort and monitoring effort to detect effort events. In an embodiment, effort may relate to a measure of strength of at least one repetitive feature in a signal. In an embodiment, effort may relate to the physical effort of a process that may affect the signal (e.g., effort may relate to work of a process). For example, effort calculated from a PPG signal may relate to the respiratory effort of a patient. Respiratory effort may increase, for example, if a patient's respiratory pathway becomes restricted or blocked. Conversely, respiratory effort may decrease as a patient's respiratory pathway becomes unrestricted or unblocked. In an embodiment, the effort of a signal may be determined by transforming the signal using a wavelet transform and analyzing features of a scalogram derived from the wavelet transform. In particular, changes in the features of the pulse band and/or breathing band in the scalogram may be correlated to a change in breathing effort.

As an additional example, the methods and systems disclosed herein may be used to determine effort in a mechanical engine. Over time, a mechanical engine may become less efficient because of wear of the mechanical parts and/or insufficient lubrication. This may cause extra strain on the engine parts and, in particular, cause the engine to exert more effort, work, or force to complete a process. Engine function may be monitored and represented using signals. These signals may be transformed and analyzed to determine effort using the techniques described herein. For example, an engine may oscillate in a particular manner. This oscillation may produce a band or bands within a scalogram. Features of this band or bands may change as the engine exerts more or less effort. The change in the features may then be correlated to effort. Methods and systems for monitoring such changes in effort to detect effort events are presented in detail below.

It will be understood that the present disclosure is applicable to any suitable signals and that PPG signals and mechanical monitoring signals are used merely for illustrative purposes. Those skilled in the art will recognize that the present disclosure has wide applicability to other signals including, but not limited to other biosignals (e.g., electrocardiogram, electroencephalogram, electrogastrogram, electromyogram, heart rate signals, pathological sounds, ultrasound, or any other suitable biosignal), dynamic signals, non-destructive testing signals, condition monitoring signals, fluid signals, geophysical signals, astronomical signals, electrical signals, financial signals including financial indices, sound and speech signals, chemical signals, meteorological signals including climate signals, and/or any other suitable signal, and/or any combination thereof.

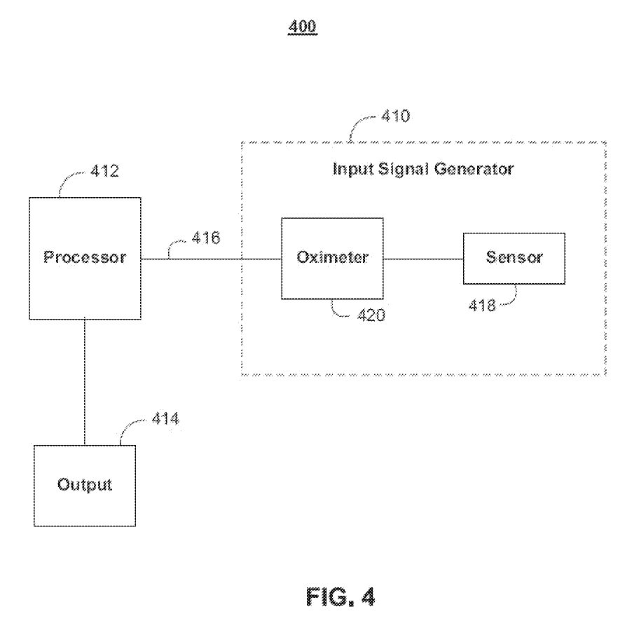

The methods for determining effort described in this disclosure may be implemented on any one or more of a multitude of different systems and apparatuses through the use of human-readable or machine-readable information. For example, the methods described herein may be implemented using machine-readable computer code and executed on a computer system that is capable of reading the computer code. An exemplary system that is capable of performing wavelet processing to determine effort is depicted in FIG. 4.

FIG. 4 is an illustrative continuous wavelet processing system in accordance with an embodiment. In an embodiment, input signal generator 410 generates an input signal 416. As illustrated, input signal generator 410 may include oximeter 420 coupled to sensor 418, which may provide as input signal 416, a PPG signal. It will be understood that input signal generator 410 may include any suitable signal source, signal generating data, signal generating equipment, or any combination thereof to produce signal 416. Signal 416 may be any suitable signal or signals, such as, for example, biosignals (e.g., electrocardiogram, electroencephalogram, electrogastrogram, electromyogram, heart rate signals, pathological sounds, ultrasound, or any other suitable biosignal), dynamic signals, non-destructive testing signals, condition monitoring signals, fluid signals, geophysical signals, astronomical signals, electrical signals, financial signals including financial indices, sound and speech signals, chemical signals, meteorological signals including climate signals, and/or any other suitable signal, and/or any combination thereof.

In an embodiment, signal 416 may be coupled to processor 412. Processor 412 may be any suitable software, firmware, and/or hardware, and/or combinations thereof for processing signal 416. For example, processor 412 may include one or more hardware processors (e.g., integrated circuits), one or more software modules, computer-readable media such as memory, firmware, or any combination thereof. Processor 412 may, for example, be a computer or may be one or more chips (i.e., integrated circuits). Processor 412 may perform the calculations associated with the continuous wavelet transforms of the present disclosure as well as the calculations associated with any suitable interrogations of the transforms. Processor 412 may perform any suitable signal processing of signal 416 to filter signal 416, such as any suitable band-pass filtering, adaptive filtering, closed-loop filtering, and/or any other suitable filtering, and/or any combination thereof.

Processor 412 may be coupled to one or more memory devices (not shown) or incorporate one or more memory devices such as any suitable volatile memory device (e.g., RAM, registers, etc.), non-volatile memory device (e.g., ROM, EPROM, magnetic storage device, optical storage device, flash memory, etc.), or both. The memory may be used by processor 412 to, for example, store data corresponding to a continuous wavelet transform of input signal 416, such as data representing a scalogram. In one embodiment, data representing a scalogram may be stored in RAM or memory internal to processor 412 as any suitable three-dimensional data structure such as a three-dimensional array that represents the scalogram as energy levels in a time-scale plane. Any other suitable data structure may be used to store data representing a scalogram.

Processor 412 may be coupled to output 414. Output 414 may be any suitable output device such as one or more medical devices (e.g., a medical monitor that displays various physiological parameters, a medical alarm, or any other suitable medical device that either displays physiological parameters or uses the output of processor 412 as an input), one or more display devices (e.g., monitor, PDA, mobile phone, any other suitable display device, or any combination thereof), one or more audio devices, one or more memory devices (e.g., hard disk drive, flash memory, RAM, optical disk, any other suitable memory device, or any combination thereof), one or more printing devices, any other suitable output device, or any combination thereof.

It will be understood that system 400 may be incorporated into system 10 (FIGS. 1 and 2) in which, for example, input signal generator 410 may be implemented as parts of sensor 12 and monitor 14, and processor 412 may be implemented as part of monitor 14.

In some embodiments, in order to determine effort, processor 412 may first transform the signal into any suitable domain, for example, a Fourier, Laplace, wavelet, Z-transform, scale, time, time-spectral, time-scale domains, a domain based on any suitable basis function, any other transform space, or any combination thereof. Processor 412 may further transform the original and/or transformed signals into any of the suitable domains as necessary.

Processor 412 may represent the original or transformed signals in any suitable way, for example, through a two-dimensional representation or three-dimensional representation, such as a spectrogram or scalogram.

After processor 412 represents the signals in a suitable fashion, processor 412 may then find and analyze selected features in the signal representation of signal 416 to determine effort. Selected features may include the value, weighted value, or change in values with regard to energy, amplitude, frequency modulation, amplitude modulation, scale modulation, differences between features (e.g., distances between ridge amplitude peaks within a time-scale band), or any combination thereof.

For example, selected features may include features in a time-scale band in wavelet space or a resealed wavelet space as described above. As an illustrative example, the amplitude or energy of the band may be indicative of the breathing effort of a patient when the band is the patient's breathing band. Furthermore, changes in the amplitude or energy of the band may be indicative of a change in breathing effort of a patient. Other time-scale bands may also provide information indicative of breathing effort. For example, amplitude modulation or scale modulation of a patient's pulse band may also be indicative of breathing effort. Effort may be correlated with any of the above selected features, other suitable features, or any combination thereof.

The selected features may be localized, repetitive, or continuous within one or more regions of the suitable domain space representation of signal 416. The selected features may not necessarily be localized in a band, but may potentially be present in any region within a signal representation. For example, the selected features may be localized, repetitive, or continuous in scale or time within a wavelet transform surface. A region of a particular size and shape may be used to analyze selected features in the domain space representation of signal 416. The region's size and shape may be selected based at least in part on the particular feature to be analyzed. As an illustrative example, in order to analyze a patient's breathing band for one or more selected features, the region may be selected to have an upper and lower scale value in the time-scale domain such that the region covers a portion of the band, the entire band, or the entire band plus additional portions of the time-scale domain. The region may also have a selected time window width.

The bounds of the region may be selected based at least in part on expected locations of the features. For example, the expected locations may be based at least in part on empirical data of a plurality of patients. The region may also be selected based at least in part on patient classification. For example, an adult's breathing band location generally differs from the location of a neonatal patient's breathing band. Thus, the region selected for an adult may be different than the region selected for a neonate.

In some embodiments, the region may be selected based at least in part on features within a scalogram. For example, the scalogram for a patient may be analyzed to determine the location of the breathing band and its corresponding ridge. A breathing band ridge may be located using standard ridge detection techniques. In an embodiment, locating a ridge may include identifying locations (a*, b*) in a scalogram which satisfy the relationship

$$\frac{\partial }{\partial a} ( \frac{{\vert T ( a , b ) \vert}^{2}}{a} ) {\vert}_{a = {a}^{*} , b = {b}^{*}} = 0 \qquad ( 23 )$$

and locations in the vicinity of the ridge of Eq. 23. Such locations may be orthogonal to the ridge of Eq. 23, and may have lower values of the quantity |T(a, b)|²/a. In an embodiment, locating a ridge may include identifying locations (a*, b*)in a scalogram which satisfy the relationship

$$\frac{\partial }{\partial a} ( {\vert T ( a , b ) \vert}^{2} ) {\vert}_{a = {a}^{*} , b = {b}^{*}} = 0 , \qquad ( 24 )$$

and locations in the vicinity of the ridge of Eq. 24. Such locations may be orthogonal to the ridge of Eq. 24, and may have lower values of the quantity |T(a, b)|².

Ridges may also be detected using the techniques described in Watson et al., U.S. application Ser. No. 12/245,326, filed Oct. 3, 2008, entitled “Systems and Method for Ridge Selection in Scalograms of Signals,” which is incorporated by reference herein in its entirety. As an illustrative example, if the ridge of a band were found to be at location X, the region may be selected to extend a predetermined distance above and below location X. Alternatively, the band itself may be analyzed to determine its size. The upper and lower bounds of the band may be determined using one or more predetermined or adaptive threshold values. For example, the upper and lower bounds of the band may be determined to be the location where the band crosses below a threshold. The width of the region may be a predetermined amount of time or it may vary based at least in part on the characteristics of the original signal or the scalogram. For example, if noise is detected, the width of the region may be increased or portions of the region may be ignored.

In some embodiments, the region may be determined based at least in part on the repetitive nature of the selected features. For example, a band may have a periodic feature. The period of the feature may be used to determine bounds of the region in time and/or scale.

The size, shape, and location of one or more regions may also be adaptively manipulated using signal analysis. The adaptation may be based at least in part on changing characteristics of the signal or features within the various domain spaces.

As a signal is being processed, for example by processor 412, the region may be moved over the signal in any suitable domain space over any suitable parameter in order to determine the value or change in value of the selected features. The processing may be performed in real-time or via a previously-recorded signal. For example, a region may move over the breathing band in the time-scale domain over time. When the selected features have been analyzed, they may be correlated with effort over time, and hence show the value or change in value of effort over time.

In some embodiments, the determined effort may be provided as a quantitative or qualitative value indicative of effort. The quantitative or qualitative value may be determined using the value or change in values in one or more suitable metrics of relevant information, such as the selected features mentioned above. The quantitative or qualitative values may be based on an absolute difference from a baseline or a calibrated value of the features. For example, breathing effort of a patient may be calibrated upon initial setup of a monitoring system. Alternatively, the values may be indicative of a relative change in the features such as the change in distance between peaks in amplitude, changes in magnitude, changes in energy level, or changes in the modulation of features.

The quantitative or qualitative value of effort may be provided to be displayed on a display, (e.g, display 28). Effort may be displayed graphically on a display by depicting values or changes in values of the determined effort or of the selected features described above. The graphical representation may be displayed in one, two, or more dimensions and may be fixed or change with time. The graphical representation may be further enhanced by changes in color, pattern, or any other visual representation.

The depiction of effort and changes in effort through a graphical, quantitative, qualitative representation, or combination of representations may be presented on output 414 and may be controlled by processor 412.

In some embodiments, a display and/or speaker on output 414 may be configured to produce visual and audible alerts, respectively, when certain effort conditions and changes in effort are detected that may represent an effort event. Visual alerts may be displayed on, for example, display 28 and audible alerts may be produced on, for example, speaker 22. In some embodiments, processor 412 may determine whether or not to produce visual, audible, or combination of alerts. The alerts may be triggered by an effort event flag, as described in detail below. Each effort event flag may result in a different visual or audible alert. In an embodiment) effort event flags may be transmitted to a printed medium, electronic storage device, or remote patient monitoring device for additional analysis and/or storage. In an embodiment, an alert may be triggered in response to a change in an effort event flag (e.g., when a patient transitions from a dangerous effort event state to a stable effort state or vice versa).

The analysis performed above that leads to a value of determined effort and/or an alert may also be used to detect events based at least in part on determined effort and/or other detected features. For example, this analysis may be used in connection with sleep studies. Increased effort may be used to detect and/or differentiate apneic events from other events. For example, reduced effort may indicate a central apnea and increased effort may indicate an obstructive apnea. Partial blockages of the upper airways may also result in an increase in effort, although air may still flow. An asthma attack may also cause an increase in effort. Post-operative respiratory issues may also result in an increase or decrease in patient respiratory effort. In an embodiment, respiration effort from a PPG signal may be used in combination with other signals typically used in sleep studies. In an embodiment, the methods disclosed herein may be used to monitor the effect of therapeutic intervention, such as the effect of a bronchodilator or other asthmatic drug on a patient's respiratory effort during an asthma attack. For example, a patient's respiratory effort may be monitored to determine how quickly the patient's respiratory effort reduces over time, if at all, after the patient receives a drug to relieve the symptoms of asthma.

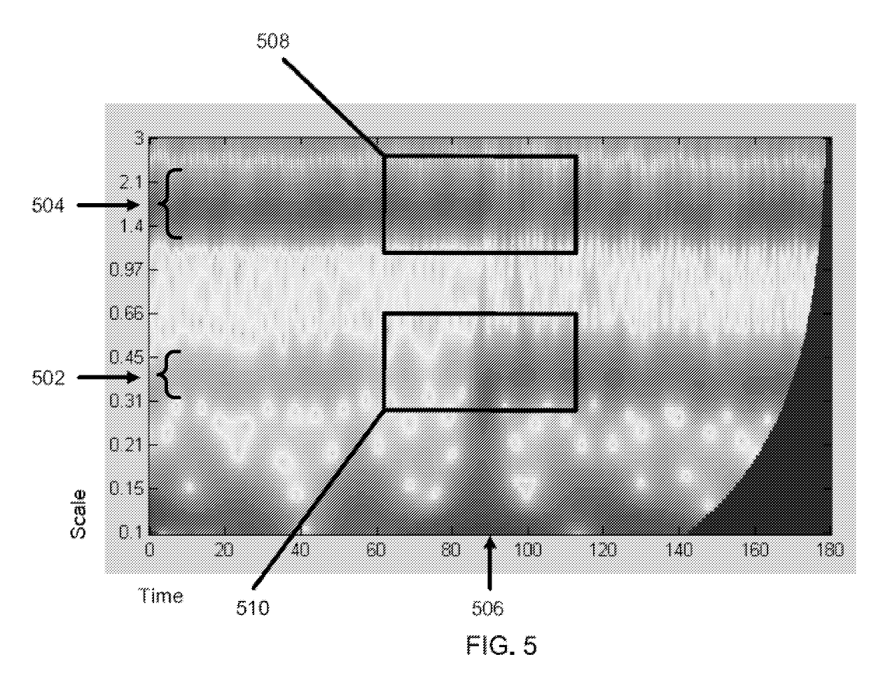

FIG. 5 shows an illustrative scalogram of a PPG signal that may be analyzed in accordance with an embodiment of the disclosure. The scalogram may be produced by system 10 of FIGS. 1 and 2 or system 400 of FIG. 4 as described above. The scalogram as shown includes breathing band 502 and pulse band 504. These bands may be identified and analyzed for features that may be indicative of breathing effort.

FIG. 5 shows an increased respiratory effort beginning at approximately time 506. This increased effort may be caused by a patient experiencing increased breathing resistance. In order to detect this change in respiration effort, regions 508 and 510 may be analyzed. A region may be characterized by a window over a portion of the scalogram. Region 508 is generally located over a portion of pulse band 504 and region 510 is generally located over a portion of breathing band 502. Regions 508 and 510 may correspond to windows that are shifted across the scalogram over time, allowing the features within the regions to be analyzed over time. The size, shape, and locations of the windows corresponding to regions 508 and 510 are merely illustrative. The features of the regions may be changed as the windows are shifted and any other suitable size, shape, and location of window may be used as described above.

At time 506, it may be observed that the modulation of the amplitudes and the modulation of the scales of pulse band 504 may begin to increase (e.g., within region 508). Analysis of this modulation or change of this modulation, as described above, may be correlated to the patients breathing effort because increased respiration effort may lead to this increase in amplitude and scale modulation of the pulse band. The modulation may be determined by analyzing, for example, the modulation of the ridge of the pulse band. The modulation of the ridge may be detected, monitored and/or analyzed using various methods, including inspecting turning points on a ridge profile with respect to time to determine one or more of scale location, amplitude and time of occurrence. This analysis may include computing an autocoltelation of the ridge profile, computing a Fourier transform of the ridge profile, performing a peak amplitude analysis, performing a secondary wavelet transform of the ridge profile, or any combination thereof.

Increased respiration effort may also lead to increased amplitude and energy of the breathing band 502. The increase in amplitude and energy can be seen within region 510 at approximately time 506. The amplitude may be determined by analyzing the ridge of the respiration band. The energy may be determined by analyzing the average or median energy within region 510. In an embodiment, the energy in a region with boundary W may be calculated in accordance with

$${\int }_{W} \int \frac{{\vert T ( a , b ) \vert}^{2}}{{a}^{2}} d a d b . \qquad ( 25 )$$

Analysis of the amplitude and/or energy or change in amplitude and/or energy within region 510 may also be correlated to the patient's breathing effort.

The patient's breathing effort may be determined based at least in part on features of one or more of the respiration band and the pulse band, such features including an amplitude modulation, a scale modulation, an amplitude, an energy, or any suitable combination thereof. A patient's breathing effort may be determined based at least in part on a change in any one or more of the features described above.

It will be understood that the above techniques for analyzing a patient's breathing effort can be used to determine any kind of effort. For example, these techniques can be used to determine the effort associated with any biological process, mechanical process, electrical process, financial process, geophysical process, astronomical process, chemical process, physical process, fluid process, speech process, audible process, meterological process, and/or any other suitable process, and/or any combination thereof.

Continuing with a previous example, the above techniques may be used to determine effort in a mechanical engine. Normal engine function may produce a band or bands within a scalogram of an engine function signal or signals. Features of this band or bands may change or become apparent as the engine exerts more or less effort. These features may include changes in the amplitude modulation, scale modulation, the amplitude, or energy of the bands. These features may also change or become apparent in other regions of the scalogram. The appearance or change in these features may then be correlated to effort or change in effort exerted by the engine.

It will also be understood that the above techniques may be implemented using any human-readable or machine-readable instructions on any suitable system or apparatus, such as those described herein.

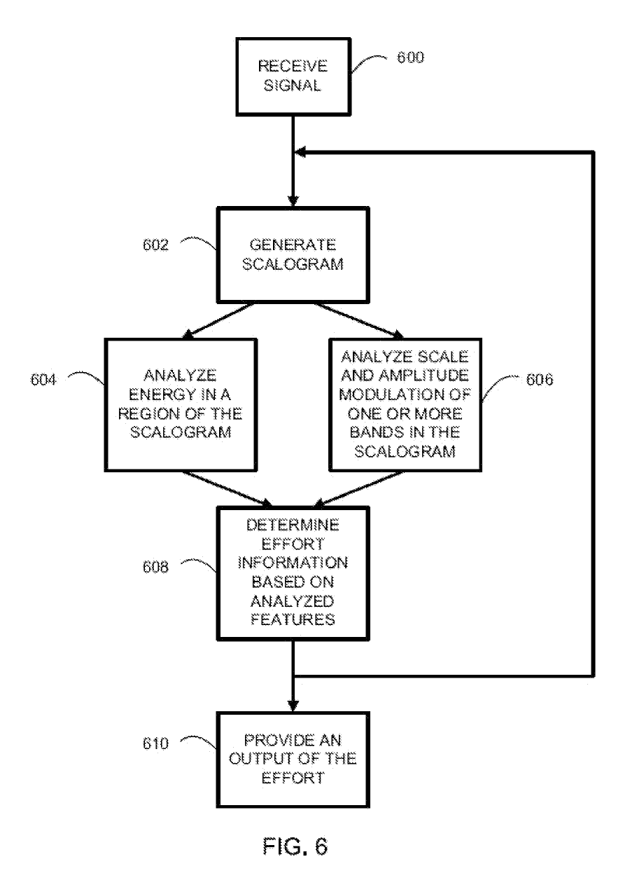

FIG. 6 is a flow chart depicting illustrative steps that may be used to determine effort. At step 600, one or more signals may be received, including any of the signals described herein, for example, one or more biosignals. As an illustrative example, the received signal may be a PPG signal.

At step 602, the received signal(s) may be transformed into any suitable domain, such as a Fourier, Laplace, wavelet, Z-transform, scale, time, time-spectral, time-scale domains, a domain based on any suitable basis function, any other transform space, or any combination thereof. For example, the signal(s) may be transformed into a time-scale domain using a wavelet transform such as a continuous wavelet transform. Once the signal is transformed into a suitable domain, it may be represented by a suitable representation. Suitable representations may include two-dimensional or three-dimensional representations. As an illustrative example, the signal may be transformed into the time-scale domain and then may be represented by a scalogram.

Once the signal is transformed, one or more features may be analyzed within the transformed signal as shown at steps 604 and 606. At steps 604 and 606, one or more regions within the transformed signal may be chosen for inspection. These regions may be similar to region 508 and region 510. As stated above with respect to region 508 and region 510, the regions may be characterized by windows of any suitable size, shape, and location. They also may be shifted across the scalogram over time, allowing features within the regions to be analyzed over time. For example, the regions may cover bands within a scalogram such as a pulse band or a respiration band. The regions may also cover any other suitable bands or features within the transformed signal.

At step 604, the features analyzed within a region may include amplitude or energy. At step 606, amplitude modulation, scale or frequency modulation, distances between peaks, and/or any other suitable features and/or combination of features may be analyzed.

At step 608, effort information may be determined based at least in part on the analysis of the features at steps 604 and 608. As described above with respect to FIG. 5, effort may be correlated with changes or the appearance of the features found and analyzed at steps 604 and 606. For example, breathing effort may be correlated with a change or weighted change in amplitude, energy, amplitude modulation, frequency modulation, and/or scale modulation in the breathing and/or pulse bands. The correlation between effort and the analyzed features may be used to determine quantitative or qualitative values associated with effort. The values may be determined based at least in part on an absolute or percentage difference from a baseline or calibrated value of effort. Furthermore, the values may be determined based at least in part on changes or appearance of the analyzed features within the signal representation. In an embodiment, an appropriate effort event flag may be triggered in response to such quantitative or qualitative values as described in additional detail below.

At step 610, the determined effort may be output. The output may be displayed on a display, such as display 28 shown in FIG. 1. A graphical display may be generated based at least in part on the determined qualitative or quantitative values representing effort or changes in effort. The graphical representation may be displayed in one, two, or more dimensions and may be fixed or change with time. The graphical representation may be further enhanced by changes in color, pattern, or any other visual representation.

As the determined effort is being output at step 610, the whole process may repeat. Either a new signal may be received, or the effort determination may continue on another portion of the received signal(s). The process may repeat indefinitely, until there is a command to stop the effort determination, and/or until some detected event occurs that is designated to halt the effort determination process. For example, it may be desirable to halt effort determination after a sharp increase in breathing effort is detected.

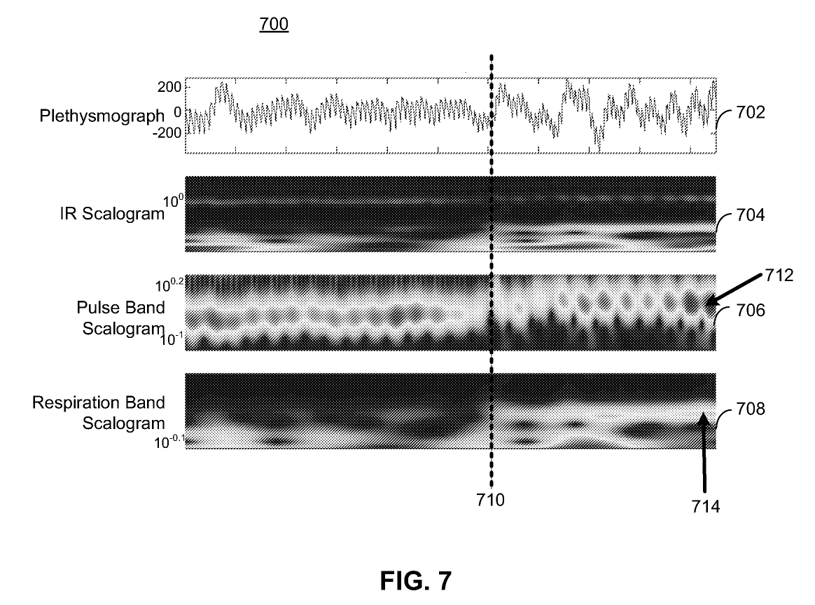

FIG. 7 depicts illustrative data 700 representative of respiratory effort. This data 700 includes an IR plethysmograph v. time waveform 702, an IR scalogram 704, a pulse band scalogram 706 and a respiration band scalogram 708. Data 700 was derived from an experiment in which a healthy male subject increased his respiratory effort at approximately 70 seconds (indicated by line 710) by breathing against a resistance The IR scalogram 704 is a wavelet transformation, as described above, of IR plethysmograph waveform 702. Two regions of IR scalogram 704 are depicted in greater detail by pulse band scalogram 706 and respiration band scalogram 708, respectively. A marked increase in energy of the breathing band can be seen in respiration band scalogram 708, commencing at approximately 70 seconds. For this subject, an increase in respiratory effort as indicated by the respiration band scalogram 708 is accompanied by a distinct increase in pulse rate as can be seen in pulse band scalogram 706.

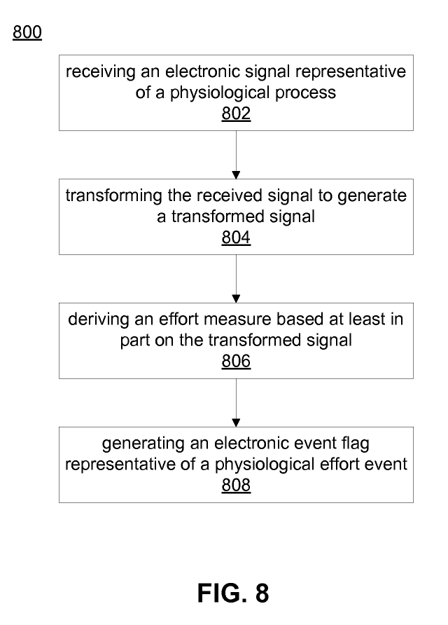

FIG. 8 is a flow chart depicting illustrative steps in a process 800 used to generate an effort event flag in accordance with an embodiment. Process 800 may be performed by processor 412, or may be performed by any suitable processing device communicatively coupled to monitor 14. Process 800 may be performed by a digital processing device, or implemented by analog hardware. At step 802, an electronic signal representative of a physiological process is received. This received signal may be generated by sensor unit 12, which may itself include any of the number of physiological sensors described herein. The received signal may be signal 416, which may be generated by oximeter 420 coupled between processor 412 and sensor 418. The received signal may include multiple signals, for example, in the form of a multi-dimensional vector signal or a frequency- or time-multiplexed signal. Additionally, the received signal received in step 802 may be a derived signal generated internally to processor 412 Accordingly, the received signal may be a transformation of a signal 416, or may be a transformation of multiple such signals. For example, the received signal may be a ratio of two signals. The received signal may be based at least in part on past values of a signal, such as signal 416, which may be retrieved by processor 412 from a memory such as a buffer memory or RAM 54. In an embodiment, the received signal may be a PPG signal, and may include at least one of a Red or IR PPG signal as discussed in detail above.

Next, at step 804, the received signal is transformed to generate a transformed signal. This transformation may be performed by any one or more of the transformation techniques described herein, including a wavelet transformation. This transformation may be performed by any suitable processing device, such as processor 412, which may itself be a general-purpose computing device or a specialized processor. In an embodiment, step 804 is based at least in part on a continuous wavelet transformation. The transformation may be selected to transform the signal into any suitable domain, for example, a Fourier, wavelet, spectral, scale, time, time-spectral, time-scale domains, or any transform space. The transformation of the received signal at step 804 may also include pre- or post-processing transformations, including filtering, compressing, and up- or down-sampling. Such filtering may, for example, smooth the received signal, take a median or other statistic of the received signal, remove erroneous regions of the received signal, or any combination thereof. The transformation may be applied to a portion or portions of the received signal. In an embodiment, a wavelet transform may be applied to a received signal, resulting in a scalogram representative of the received signal. The transformation of step 804 may be broken into one or more stages performed by one or more devices within wavelet processing system 400 (which may itself be a part of effort system 10). For example, a transformation of a signal received at sensor 12 may be filtered by low pass filter 68 prior to undergoing additional processing at microprocessor 48 within effort system 10.

At step 806, an effort measure is derived based at least in part on the transformed signal. As described above, an effort signal may be derived using any feature or group of features in the wavelet-transformed data representative of a physical process. The effort measure derived at step 806 may include any one or more of the transformed data features described herein. In an embodiment, the derived effort signal may be a respiratory effort signal, which may communicate information regarding changes in respiratory effort that may indicate a respiratory effort event. The effort signal may be related to any of a number of relevant features in the wavelet-transformed data, and may be derived in accordance with any of the embodiments described above. The effort measure may be derived by any suitable processor, such as microprocessor 48, or may be extracted by special-purpose analog hardware.

In an embodiment, the effort signal may be determined by summing scalogram values within a portion of the scalogram, such as a confined locality of a band of interest, (e.g., the breathing band of a PPG scalogram). In an embodiment, the effort signal may be determined by determining the increase in energy manifest as increasing respiratory sinus arrhythmia, or any relevant variation in pulse rate or other physiological signal that occurs over a breathing cycle. In an embodiment, the effort signal may be determined by summing the increase in energy manifest as the pulse amplitude of a ridge of a band of interest in the scalogram, (e.g., the respiration band of a PPG scalogram).

During an effort event, a band of interest may exhibit a change in scale over time. For example, the respiration band of a PPG scalogram may shift when a monitored patient exerts additional respiratory effort (e.g., as illustrated in FIG. 3(d)). In an embodiment, the effort signal may be determined by identifying and tracking features of the wavelet transform which correspond to changes in band position. An increase or decrease in respiratory rate and/or heart rate may cause a change in band position. Such respiratory rate and/or heart rate changes may be caused by, for example, heart conditions (e.g., tachycardia, bradycardia), administration of a stimulating drug (e.g., adrenalin), the administration of a relaxation-inducing drug (e.g., an opiate), exercise, psychological stress, natural relaxation, or any combination thereof. An increase in respiratory effort may itself trigger an associated change in respiration rate and/or heart rate, and may correspond to a change in band position.

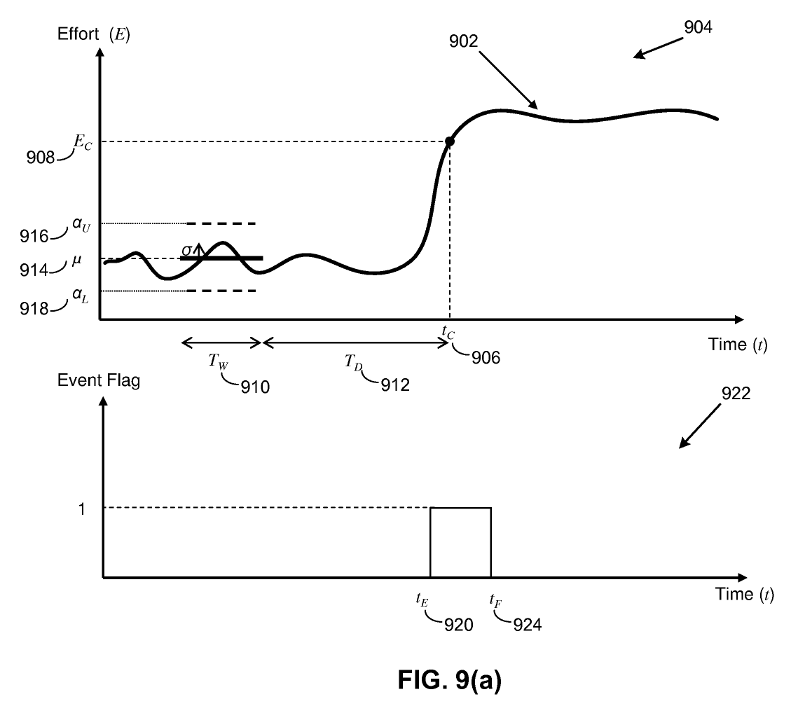

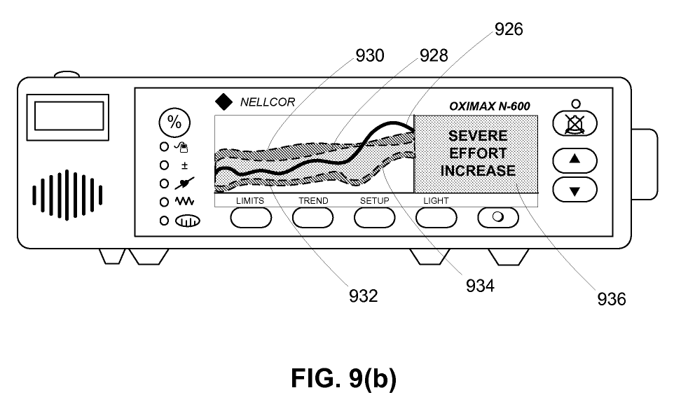

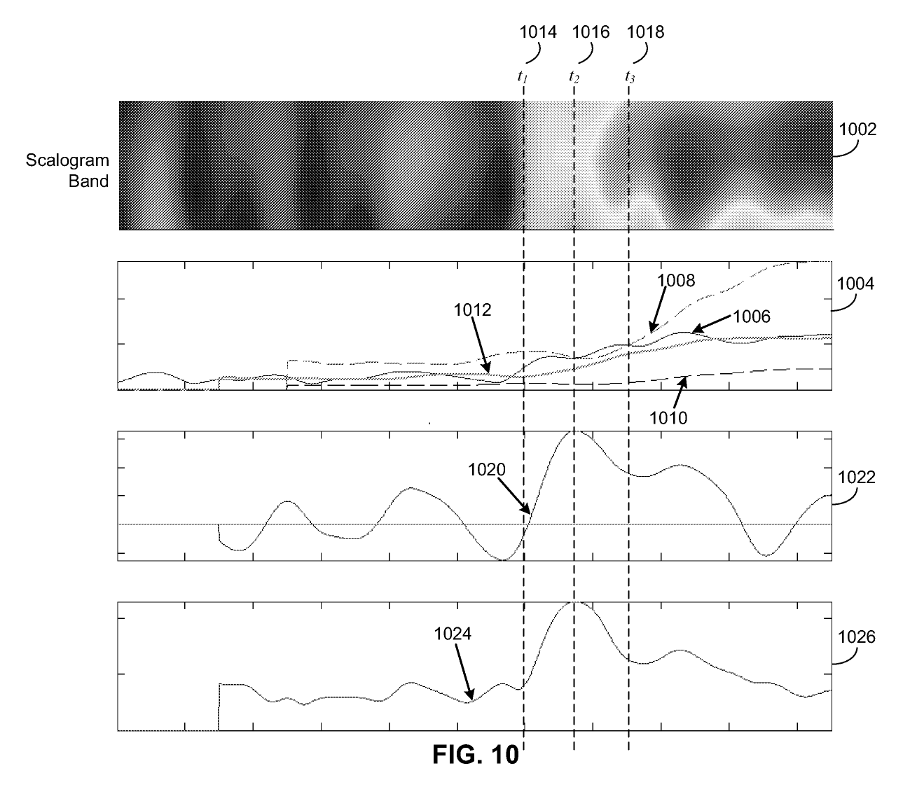

At step 808, an electronic event flag representative of a physiological effort event is generated based at least in part on a comparison between the derived effort measure and at least one reference effort measure. In an embodiment, an electronic event flag representative of a respiratory effort event is generated based at least in part on a comparison between a respiratory effort measure derived at step 806 and at least one reference respiratory effort measure. The electronic event flag may be generated, for example, by processor 412 and transmitted to output 314. Output 314 may represent an indicator device such as displays 20 and 28, speakers 22 and 30, a paper or physical recording device, an electronic memory such as RAM 54, or any combination thereof. The electronic event flag generated at step 808 may take any suitable form for communication of effort event information to a device, patient or care provider. Illustrative embodiments of step 808 are described in additional detail below, with reference to FIGS. 9-10.

FIG. 9(a) depicts an illustrative effort signal 902 and an effort event determination process in accordance with an embodiment. The particular shape of the effort signal 902 depicted in plot 904 is simply illustrative; an effort signal may be calculated in accordance with any of the techniques described herein. Plot 904 also depicts a number of features of effort signal 902 that may be used to generate an effort event flag. For example, at time point t_{C} 906, the corresponding effort signal value E_{C} 908 may be determined. Effort signal value E_{C} 908 may then be compared against a reference effort measure. In an embodiment, the reference effort measure may be a measure derived from past values of effort signal 902. These past values may be values arising over a time window or windows. Generally, the term “time window” may be used to refer to one or more intervals of time, a number of periods in a signal with periodic features, or a combination thereof. For example, effort signal value E_{C} 908 may be compared against a measure taken over a time window of length T_{W} 910 located at a time delay T_{D} 912 prior to t_{C} 906. In an embodiment, the length T_{W} 910 may be chosen to roughly correspond to an integer number of breaths taken by a patient. The measure taken over a time window may include any of a mean value) a weighted mean value, a median value, a maximum value, a minimum value, a gradient value, a standard deviation value, or any of a number of measures described herein and described in additional detail below. In an embodiment, a reference effort measure may be a measure derived from substantially all past values of the effort signal 902, and may be based at least in part on any of the above measures. In an embodiment, the reference effort measure may be a fixed value, or may be based on patient-specific information such as age, weight, gender, health status, any other relevant criterion, or any combination thereof. Reference error measures based on past values of an effort signal may be advantageously applied to effort signals that vary considerably from patient to patient and across time, and may improve the accuracy of effort event detectors by making useful assessments of relative effort.

In an embodiment, a comparison between a derived effort measure (e.g, the effort signal 902) and a reference error measure may take the form of a threshold test. Generally, a threshold test on a value may test any of a number of threshold conditions, including whether the value exceeds a single threshold, whether the value is below a single threshold, or whether the value falls within a specified range or ranges. A threshold test may be fixed, and retrieved by processor 312 from ROM 52 or RAM 54. A threshold test may be dynamic and depend, for example, on past values of a received or derived signal. A threshold test may also depend on signal quality indicators, such as an indicator arising from an electromagnetic noise measuring device or a signal arising from sensor 418 indicating a malfunction or undesirable operating condition. In such an embodiment, an indicator of low signal quality may result in adjusting the parameters of a threshold test to reduce the possibility of false alarm or a missed effort event.

In an embodiment, thresholds may be set at points above a reference effort measure, below a reference effort measure, substantially equal to a reference effort measure, or any combination thereof. These thresholds may define a range or ranges of values within which the effort signal may fall. For example, FIG. 9(a) illustrates a reference effort measure μ 914, which is the mean value calculated over the illustrated window of length T_{W} 910. Upper threshold α_{U} 916 and lower threshold α_{L} 918 may be set. Upper and lower thresholds may be located at equal intervals from the reference effort measure, or may be located at unequal intervals. Thresholds may vary in time, and may be based at least in part on effort signal 902. In an embodiment, a threshold may be set at a multiple of the standard deviation of the effort signal over a time window above or below a reference effort value. In an embodiment, a threshold may be set as a multiple or fraction of the mean of the effort signal over a time window.

In an embodiment, multiple thresholds may be set. Each of these multiple thresholds may indicate a different severity or nature of an effort event. Each of these multiple thresholds may trigger a corresponding effort event flag, which may have differing values. A threshold test may include one or more upper thresholds, one or more lower thresholds, or a combination thereof. Thresholds may be set based on any number of factors, including features of the effort signal, signal quality indicators, and patient-specific information. Factors that may influence the setting of thresholds are discussed in additional detail below.

The results of a threshold test may trigger an effort event flag. For example, FIG. 9(a) illustrates an effort event flag triggered at time t_{E} 920 when effort signal 902 exceeds upper threshold α_{U} 916. The triggering of the event flag is indicated in plot 922 of FIG. 9(a) by an event flag value of “1.” In an embodiment, generating an event flag may include setting an event flag variable equal to a specified value or values in a memory (e.g., RAM 54). In an embodiment, generating an event flag may include generating a logic signal that may be passed directly to an output. In an embodiment, generating an event flag may include generating a signal to be transmitted which encodes the result of a threshold test in an amplitude, frequency, duty cycle, waveform shape, or other feature of a signal. At time t_{F} 924, the value of the event flag is set back to “0,” which may indicate the end of a respiratory event or a return to an nominal patient state. Resetting the event flag to “0,” or performing any adjustment of an event flag or flags, may be triggered by a threshold test or tests as described above. As is discussed in additional detail below, in an embodiment, one or more different flags may be generated to indicate one or more types of effort events. It will be understood that the triggering of an event flag in response to an effort signal exceeding an upper threshold, as illustrated in FIG. 9(a), is simply an example of an effort event determination process. Many other such processes are within the scope of this disclosure. For example, an event flag may be triggered when an effort signal decreases below a lower threshold (such as lower threshold α_{L} 918), which may indicate a decrease in effort and signal an effort event. An effort event flag may be suitably triggered whenever a significant change in effort is detected.

The sensitivity and performance of an effort event detection process may be adjusted by, for example, changing the form and parameters of effort event flag threshold tests. In an embodiment, the sensitivity and performance of the process illustrated in FIG. 9(a) and described above may be adjusted by changing one or more of the parameters such as α_{U} 916, α_{L} 918, T_{W} 910, and T_{D} 912. Threshold conditions which trigger an effort event flag may be determined by past measurements of a patient's physiological signals, expected statistical distributions of physiological signals, analytical or theoretical models of physiological function, empirical or observational data of physiological signals of a population, or any combination thereof. In an embodiment, an EEG signal may be monitored in conjunction with an effort signal, for example, during sleep studies to study the effect of arousals in the apnea process. In an embodiment, an EMG signal may be monitored in conjunction with an effort signal, for example, to compare breathing effort with related muscular activity. In an embodiment, a pulse oximetry signal may be monitored in conjunction with an effort signal, for example, to monitor a patient's ability to absorb oxygen during the respiratory process. In such an embodiment, an increase in effort together with a decrease in blood oxygen saturation may indicate, for example, that the patient is physiologically compromised and may coincide with an increase in respiratory rate. Additional signals that may be monitored in conjunction with an effort signal include respiratory rate, pulse rate, blood pressure, any other physiological signal, or any combination of the signals described herein.

In an embodiment, the threshold values may be based at least in part on patient data. Examples of patient data include body mass index, lung capacity, and any physiological characteristic. For example, obese patients may require an increased effort to breathe, which may correspond to a correlation between body mass index and a baseline respiratory effort. Such a correlation may be used to adjust or set a suitable threshold respiratory effort value during patient monitoring.

FIG. 9(b) depicts an illustrative effort monitoring system display screen in accordance with an embodiment. This display screen is depicted as embedded within a unit similar to monitor 14 of FIG. 2, but it will be understood that this screen is merely illustrative and could be included in the display of any device coupled to output 414 as discussed above.

In the embodiment illustrated in FIG. 9(b), effort waveform 926, first upper threshold 928, second upper threshold 930, first lower threshold 932 and second lower threshold 934 may be displayed. Effort waveform 926 and thresholds 928-934 may be communicated to output 414 by the processor 412, and may be derived by any of the techniques and devices described herein. In an embodiment, first upper threshold 930 may correspond to the onset of an increased effort event, while second upper threshold 932 may correspond to the onset of a severe increased effort event. In an embodiment, first lower threshold 932 may correspond to the onset of a decreased effort event, while second lower threshold 934 may correspond to the onset of a severe decreased effort event. The regions between the thresholds may be visually indicated to allow a patient or care provider to quickly compare effort waveform 926 to thresholds 928-934.

The display of FIG. 9(b) also includes an alert message 936 alerting the care provider that effort waveform 926 has exceeded a threshold, signifying an effort event. In the illustrated example, effort waveform 926 has exceeded second upper threshold 930, indicating the onset of a severe increased effort event. In an embodiment, an alert may be based on the effort signal and one or more other monitored physiological signals.

FIG. 10 depicts an illustrative band of a scalogram 1002 and several effort measures that may be used to determine an effort event in accordance with an embodiment. In an embodiment, scalogram 1002 may be the respiration band of a scalogram derived from a PPG signal. Plot 1004 depicts effort signal 1006, upper threshold 1008, lower threshold 1010 and reference effort measure 1012. Effort signal 1006 may be calculated by summing the scalogram values across a band region such as that illustrated in scalogram 1002 (e.g., corresponding to the respiration band, pulse band, or any particular band of interest). Commencing at approximately time t₁ 1014, an increase in energy in scalogram 1002 is apparent, and may be reflected in the rise of the effort signal 1006 in plot 1004, commencing at roughly the same time t₁ 1014. Plot 1004 also depicts reference effort measure 1012, which may be computed in accordance with any of the embodiments described above. For example, reference effort measure 1012 may be calculated as the mean of previous effort values over a window as described with reference to FIG. 9(a). Upper threshold 1008 and lower threshold 1010 may be computed in any suitable manner described herein, including multiples of a mean value or a reference effort measure (e.g., 2.5 and 0.4 of reference effort measure 1012, respectively). As described with reference to FIG. 9(a), in an embodiment, an effort event flag may be triggered when the effort signal 1006 exceeds a threshold; for example, in FIG. 10, effort signal 1006 exceeds upper threshold 1008 between times t₂ 1016 and t₃ 1018.

In an embodiment, an effort event flag may be triggered in response to a result of a threshold test on a measure derived from the effort signal. Such a derived measure may allow improved detection of effort events by isolating and identifying patterns or signals of particular interest within the effort signal. In an embodiment, the derived measure may be the area under an effort signal above a predefined value. In an embodiment, the derived measure may be the amount of time that an effort signal spends above a predefined value. Such a derived measure may be used to indicate a characteristic time scale of an effort change.

In an embodiment, the derived measure may be a smoothed gradient of the effort signal over a window or windows. A smoothed gradient measure 1020 is illustrated in plot 1022 of FIG. 10. Such a smoothed gradient may be calculated, for example, by finding the slope of a line fit to the effort signal over a window. This line may be a best-fit line to the effort signal within the window, and may be determined by any of a number of line-fitting techniques, including ordinary least squares, total least squares, algebraic and geometric techniques. In an embodiment, a smoothed gradient may be calculated by any of a number of gradient determination and approximation techniques, including those suitable for sampled data (e.g., forward difference, backward difference, central difference, higher-order methods, and any automatic differentiation method). In an embodiment, the derived measure may be an area under the best fit gradient line between zero crossings, which may be indicative of the severity of an effort change.

In an embodiment, the derived measure may be a standard deviation of the effort signal over a window or windows. A large standard deviation suggests a wide spread of data, which may be indicative of a sudden change in effort. A standard deviation measure 1024 is illustrated in plot 1026 of FIG. 10. In an embodiment, the derived measure may be an area under a standard deviation curve above a pre-defined threshold value. While the standard deviation measure does not contain the polarity associated with negative and positive changes in effort (as does, for example, a gradient measure, which can take both positive and negative values), either or both measures may be used when determining whether an effort event has occurred. Any such measure of variability and/or dispersion may also be used, including, for example, a variance, an entropy, and an index of variability.

In an embodiment, a derived measure may be the result of applying a filtering operation to an effort signal. Filtering may be performed on an analog or digital representation of an effort signal, and may be performed in hardware or software. This filtering operation may result in a derived measure that is substantially similar to a derived measure obtained by another means, such as a smoothed gradient technique (e.g., a high-pass filter may provide a gradient determination). In an embodiment, a derived measure is based at least in part on an FIR filter, an IIR filter, or a combination of the two.

In an embodiment, an effort signal or a measure derived from an effort signal may be provided along with a triggered flag to provide additional information regarding a detected effort event. In an embodiment, an effort signal or a measure derived from the effort signal may be used to “weight” a triggered flag, for example, when additional analysis on the triggered flag is to be performed. In this manner, information about patient effort contained in both the flag and the effort signal may be used for further analysis (e.g., when training a neural network or other patient status prediction algorithm).

In an embodiment, a measure of variability of the effort signal (e.g., the standard deviation calculated over a time window) may be used as a measure of the stability of the effort signal, reliability of the effort signal, noise content of the effort signal, or any combination thereof. Such measures may be used to adjust the parameters of a threshold test for triggering effort event flags in response to fluctuations in the effort signal. For example, an upper threshold may be set according to a multiple of the mean of the effort signal plus a multiple of the standard deviation of the effort signal over the window to account for effort signals with more inherent fluctuations, which may be due to artifact, noise, respiratory activity, patient movement, or additional factors. Additionally, such a measure may be used to “weight” a triggered flag in the manner discussed above.

In an embodiment, the threshold test for triggering a subsequent event flag may be based on current or past values of the effort event flag. In an embodiment, a Schmitt tigger may be used to trigger and reset an effort event flag. For example, an effort event flag may be triggered when the effort signal is greater than a first deviation from a nominal value, and may not be reset until the effort signal drops below a value that is less than a second deviation from the nominal value. In an embodiment, the threshold for a second positive event flag may be higher or lower than the threshold for a first positive event flag (and analogously for negative event flags). For example, a first threshold may be set for a first positive event flag to indicate the onset of a respiratory event. A second positive event flag, which may indicate an increase in the severity of the respiratory event, may be triggered when the effort signal exceeds a second threshold that represents a smaller increase in effort than was required to trigger the first positive event flag. Such a trigger allows for adjustable sensitivity of the event flags to different ranges of the effort signal, which may correspond to more or less critical patient conditions.

In an embodiment, a threshold test may include a time component that may be satisfied before an effort event flag is triggered. For example, an effort signal or derived measure may cross a threshold briefly due to transient artifact, without indicating the onset of a true effort event. In an embodiment, a threshold may be required to be crossed for a predetermined length of time before triggering the flag. This length of time may depend on the effort signal, a derived measure, or any other source of patient status information relevant to effort event detection. Such an embodiment may advantageously mitigate against triggering due to transient artifacts of limited time duration.

In an embodiment, the effort signal may be monitored in conjunction with another signal to provide information on patient effort or status, to trigger an effort event flag, or both. Examples of signals that may be monitored in conjunction with the effort signal include an amplitude modulation of the pulse band, a respiratory sinus arrhythmia, a breathing rate, a pulse rate, a blood pressure, an oxygen saturation signal a chest or abdomen motion signal, a respiratory tract pressure, a temperature, and a pulse transit time. Monitoring a signal in conjunction with the effort signal may involve calculating a correlation between the two signals, which may be used to provide an improved estimate of the patient effort, determine a confidence in the patient effort measurement, reduce noise in the patient effort measurement, validate a patient effort measurement, or any combination thereof. In an embodiment, an effort event flag is triggered based on a calculated correlation between the effort signal and another monitored signal. For example, pulse transit time may be used as an indicator of microarousal and apneic events, and a pulse transit time signal may exhibit similar structural features to an effort signal based on the respiration rate of a PPG signal.

In an embodiment, an effort event flag is triggered based on both a threshold test on an effort signal and a threshold test on another monitored signal. For example, a motion sensor signal may detect patient movement. If the detected movement exceeds a threshold level, an effort event flag may not be triggered until patient movement returns to a tolerable level. Such an embodiment may help prevent false effort event flags due to patient motion artifacts in the effort signal. A threshold test on an effort signal may be based on another monitored signal. The value of T_{D}, T_{W}, or both, may be adjusted during the monitoring of a patient. Such dynamic adjustment may be based on the respiration rate. In an embodiment, a respiration rate may be used to determine suitable values for the parameters in a threshold test on the effort signal. For example, the value of T_{D}, T_{W}, or both, may be based on the respiration rate to provide for variable length windows and delays to capture a respiratory effort event. In an embodiment, the value of T_{W} may be increased to a value greater than the period of a single breath. In such an embodiment, the effort variations within each breath cycle may be smoothed out, which may allow a patient or care provider to focus on longer term trends of increased effort over a number of breaths.

In an embodiment, the timing of an event or events may be based on the time elapsed between two event flags. For example, the time between a positive event flag (which may signal the onset of an effort event) and the next negative event flag (which may signal the end of an effort event) may be taken as the duration of the effort event. In an embodiment with multiple positive event flags, multiple negative event flags, or any combination of the two, the duration of a respiratory event may be measured by the time elapsed over any suitable sequence of event flags. For example, a first positive event flag may be triggered when the effort signal exceeds a first threshold, and a second positive event flag may be triggered when the effort signal exceeds a second, higher threshold. In this example, the duration of an overall effort event may be calculated from the first positive event flag, while the duration of a severe effort event may be calculated from the second positive event flag. In an embodiment, the time elapsed between two or more positive event flags or two or more negative event flags may be used to indicate a rate of change in patient condition (e.g., a relatively slow or relatively rapid deterioration or recovery). Such a rate of change may indicate a severity of a patient condition.

In an embodiment, an effort event flag may be used as a signal representative of a patient's physiological condition. The waveform associated with an effort event flag monitored over time may be analyzed using any of the signal analysis techniques described herein, and may be indicative of changes in effort associated with a physiological condition. For example, different variants of sleep apnea disorders may result in different patterns of respiratory effort increase and decrease. Such patterns may be captured by monitoring and analyzing an effort event flag over time. In an embodiment, a measure of variability or dispersion may be derived from the effort event flag waveform to assess the frequency of changes in a patient's physiological state, the severity of changes in a patient's physiological state, or a combination of the two. A measure of variability may include a standard deviation, a variance, an entropy, an index of variability, or any combination thereof. For example, changes in an effort event flag waveform may be measured as an index of variability in accordance with

$$1 - {\sum }_{i = 1}^{N} {( {p}_{i} )}^{2} , \qquad ( 26 )$$

in which N represents the number of occurrences of an event flag waveform and pᵢ represents the probability of occurrence of the i th particular value of the event flag waveform.

In an embodiment, one or more effort event flags may be used to characterize the respiration of a sleeping patient. In such an embodiment, regular apnea cycles may exhibit particular patterns of effort event flags. For example, two effort event flags may be used to indicate a distinct rise and fall in effort, respectively. Such effort event flags may alternate when a patient exhibits a cyclical apnea pattern of airflow cessation and resumption (e.g., with cycles on the order of approximately one minute). For an apneic patient, an effort signal may exhibit a cyclical morphology. The cyclical pattern of behaviour of an effort signal and/or an effort event flag signal may be characterized using an autocorrelation function, frequency spectrum, or other transformation, including a wavelet transform. In an embodiment, the characterization of a cyclical pattern may be used to detect or diagnose an apneic condition (e.g., by applying a threshold test).

It will also be understood that the above method may be implemented using any human-readable or machine-readable instructions on any suitable system or apparatus, such as those described herein.

The foregoing is merely illustrative of the principles of this disclosure and various modifications can be made by those skilled in the art without departing from the scope and spirit of the disclosure. The following claims may also describe various aspects of this disclosure.

What is claimed is:

1. A method for monitoring respiratory effort, comprising:
receiving an electronic photoplethysmograph signal;
transforming the photoplethysmograph signal into a transformed signal based at least in part on a continuous wavelet transform;
deriving, using processing equipment, a respiratory effort measure based at least in part on the transformed signal; and
generating an electronic event flag representative of a respiratory effort event based at least in part on a comparison between the respiratory effort measure and at least one reference respiratory effort measure.

2. The method of claim 1, wherein the at least one reference respiratory effort measure is based at least in part on past instances of the respiratory effort measure within a time window.

3. The method of claim 2, wherein the at least one reference respiratory effort measure is based at least in part on a smoothed gradient of the past instances of the respiratory effort measure within the time window.

4. The method of claim 2, wherein the at least one reference respiratory effort measure is based at least in part on an area under the past instances of the respiratory effort measure within the time window.

5. The method of claim 2, wherein the at least one reference respiratory effort measure is based at least in part on a mean value of the past instances of the respiratory effort measure within the time window.

6. The method of claim 1, wherein generating an electronic event flag comprises:
applying a threshold test to the derived effort measure to generate a result, wherein the threshold test is based at least in part on the at least one reference effort measure; and
generating an electronic event flag based at least in part on the result.

7. The method of claim 6, wherein applying the threshold test comprises comparing the derived effort measure against multiple thresholds.

8. The method of claim 7, wherein the multiple thresholds are based at least in part on the variability of the respiratory effort measure.

9. The method of claim 6, wherein the threshold test is based at least in part on at least one of current and past occurrences of the electronic event flag.

10. The method of claim 6, wherein the threshold test is based at least in part on a physiological signal that is not the respiratory effort measure.

11. A system for monitoring respiratory effort, comprising:
at least one memory device;
an indicator device, capable of indicating a respiratory effort event in response to an event flag; and
a processor, communicably coupled to the at least one memory device and the indicator device and capable of receiving an electronic photoplethysmograph signal, the processor being capable of:
calculating a transformed signal based at least in part on at least on the electronic photoplethysmograph signal and a continuous wavelet transform;
deriving a respiratory effort measure based at least in part on the transformed signal; and
generating an event flag representative of a respiratory effort event based at least in part on a comparison between the respiratory effort measure and at least one reference respiratory effort measure

12. The system of claim 11, wherein the at least one reference respiratory effort measure is based at least in part on past instances of the respiratory effort measure within a time window.

13. The system of claim 12, wherein the at least one reference respiratory effort measure is based at least in part on a smoothed gradient of the past instances of the respiratory effort measure within the time window.

14. The system of claim 12, wherein the at least one reference respiratory effort measure is based at least in part on an area under the past instances of the respiratory effort measure within the time window.

15. The system of claim 12, wherein the at least one reference respiratory effort measure is based at least in part on a mean value of the past instances of the respiratory effort measure within the time window.

16. The system of claim 11, wherein generating an event flag comprises:
applying a threshold test to the derived effort measure to generate a result, wherein the threshold test is based at least in part on the at least one reference effort measure; and
generating an event flag based at least in part on the result.

17. The method of claim 16, wherein the multiple thresholds are based at least in part on the variability of the respiratory effort measure.

18. The method of claim 16, wherein the threshold test is based at least in part on at least one of current and past occurrences of the electronic event flag.

19. Computer-readable medium for use in monitoring patient effort, the computer-readable medium having computer program instructions recorded thereon for:
receiving an photoplethysmograph signal;
transforming the photoplethysmograph signal into a transformed signal based at least in part on a continuous wavelet transform;
deriving a respiratory effort measure based at least in part on the transformed signal; and
generating an event flag representative of a respiratory effort event based at least in part on a comparison between the respiratory effort measure and at least one reference respiratory effort measure.

20. The computer-readable medium of claim 19, wherein the at least one reference respiratory effort measure is based at least in part on past instances of the respiratory effort measure within a time window.
